(19) United States
(12) Patent Application Publication
(10) Pub. No.: US 2005/0014788 A1
(43) Pub. Date: Jan. 20, 2005

(54) Piperidine derivatives and their use as modulators of chemokine receptor activity (especially ccr5)

(76) Inventors: John Cumming, Cheshire (GB); Howard Tucker, Cheshire (GB)

Correspondence Address: FISH & RICHARDSON PC, 225 FRANKLIN ST, BOSTON, MA, 02110, US

(21) Appl. No.: 10/495,405

(22) PCT Filed: Nov. 12, 2002
(86) PCT No.: PCT/SE02/02054

(30) Foreign Application Priority Data
Nov. 15, 2001 (SE) 0103819-9

(51) Int. Cl. A61K 31/454; A61K 31/445; C07D 211/56

(52) U.S. Cl. 514/317; 514/326; 546/326; 546/224

(57) ABSTRACT

Compounds of formula (I): compositions comprising them, processes for preparing them and their use in medical therapy (for example modulating CCR5 receptor activity in a warm blooded animal).

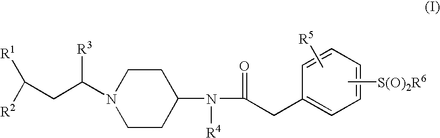

The present invention relates to heterocyclic derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in PCT/SE01/01053, EP-A1-1013276, WO00/08013, WO99/38514 and WO99/04794.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important rôle in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or α) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cel expressed and secreted” (RANTES), macrophage inflammatory proteins (MIP) MIP-1α and MIP-1β and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

The present invention provides a compound of formula (I): 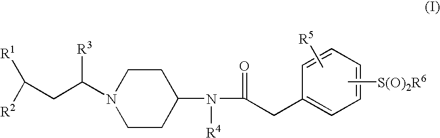
wherein:
R¹ is phenyl {para-substituted by: halo, hydroxy, nitro, S(O)ₖ(C₁₋₆alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, C(O)[N-linked heterocyclyl], CO₂H, CO₂(C₁₋₆ alkyl), NHC(O)(C₁₋₆ alkyl), NHC(O)O(C₁₋₆ alkyl), NHS(O)₂(C₁₋₆ alkyl), C(O)(C₁₋₆ alkyl), CF₃, OCF₃, phenyl, heteroaryl, (C₁₋₄ alkyl)phenyl, (C₁₋₄ alkyl)heteroaryl, NHC(O)phenyl, NHC(O)heteroaryl, NHC(O)(C₁₋₄ alkyl)phenyl, NHC(O)(C₁₋₄ alkyl)heteroaryl, NHS(O)₂phenyl, NHS(O)₂heteroaryl, NHS(O)₂(C₁₋₄ alkyl)phenyl, NHS(O)₂(C₁₋₄ alkyl)heteroaryl, NHC(O)NH(C₁₋₆ alkyl), NHC(O)NH(C₃₋₇ cycloalkyl), NHC(O)NHphenyl, NHC(O)NHheteroaryl, NHC(O)NH(C₁₋₄ alkyl)phenyl or NHC(O)NH(C₁₋₄ alkyl)heteroaryl; wherein the foregoing phenyl and heteroaryl groups are optionally substituted by halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃};
R² is phenyl or heteroaryl, either of which is optionally substituted by halo, C₁₋₄ alkyl, C₁₋₄ alkoxy, S(O)ₙ(C₁₋₄ alkyl), nitro, cyano or CF₃;
R³ is hydrogen or C₁₋₄ alkyl;
R⁴ is ethyl, allyl or cyclopropyl;
R⁵ is hydrogen, halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
R⁶ is C₁₋₄ alkyl;
k, m and n are, independently, 0, 1 or 2;
or a pharmaceutically acceptable salt thereof or a solvate thereof;
   provided that:
   when R³ and R⁵ are both hydrogen, R⁴ is ethyl, R⁶ is para-(S(O)₂CH₃) and R² is unsubstituted phenyl then R¹ is not para-methoxy-phenyl, para-methyl-phenyl, para-trifluoromethyl-phenyl or 3,4-dichlorophenyl;
   when R³ and R⁵ are both hydrogen, R⁴ is ethyl, R⁶ is para-(S(O)₂CH₃) and R² is unsubstituted phenyl, pyrid-2-yl or pyrid-4-yl then R¹ is not para-chloro-phenyl; and,
   when R³ and R⁵ are both hydrogen, R⁶ is para-(S(O)₂CH₃) and R² is meta-chloro-phenyl, unsubstituted phenyl or thiophen-3-yl then R¹ is not para-fluoro-phenyl.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts (adducts) such as a hydrochloride, hydrobromide, phosphate, acetate, fumarate, maleate, tartrate, citrate, oxalate, methanesulphonate or p-toluenesulphonate, or, additionally, formate. Acid addition salt is, for example hydrochloride or formate.

The compounds of the invention may exist as solvates (such as hydrates) and the present invention covers all such solvates.

Alkyl groups and moieties are straight or branched chain and are, for example, methyl (sometimes abbreviated to Me), ethyl, n-propyl, iso-propyl, n-butyl, sec-butyl or tert-butyl.

Cycloalkyl is for example, cyclopropyl, cyclopentyl or cyclohexyl.

N-Linked heterocyclyl is a nitrogen-linked, non-aromatic 3, 4, 5 or 6 membered ring optionally comprising one further heteroatom (selected from the group comprising nitrogen, oxygen and sulphur). It is, for example, aziridinyl, azetidinyl, pyrrolidinyl, piperidinyl, piperazinyl, morpholinyl or thiomorpholinyl.

Heteroaryl is an aromatic 5 or 6 membered ring, optionally fused to one or more other rings, comprising at least one heteroatom selected from the group comprising nitrogen, oxygen and sulphur; or an N-oxide thereof, or an S-oxide or S-dioxide thereof. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,4]-triazolyl, pyridinyl, pyrimidinyl, indolyl, benzo[b]furl (also known as benzfuryl), benz[b]thienyl (also known as benzthienyl or benzthiophenyl), indazolyl, benzimidazolyl, benztriazolyl, benzoxazolyl, benzthiazolyl, 1,2,3-benzothiadiazolyl, an imidazopyridinyl (such as imidazo[1,2a]pyridinyl), thieno[3,2-b]pyridin-6-yl, 1,2,3-benzoxadiazolyl (also known as benzo[1,2,3]thiadiazolyl), 2,1,3-benzothiadiazolyl, benzofurazan (also known as 2,1,3-benzoxadiazolyl), quinoxalinyl, a pyrazolopyridine (for example 1H-pyrazolo[3,4-b]pyridinyl), quinolinyl, isoquinolinyl, a naphthyridinyl (for example [1,6]naphthyridinyl or [1,8]naphthyridinyl), a benzothiazinyl or dibenzothiophenyl (also known as dibenzothienyl); or an N-oxide thereof, or an S-oxide or S-dioxide thereof. Heteroaryl is especially pyridyl, pyrimidinyl; indolyl or benzimidazolyl.

(C₁₋₄ Alkyl)phenyl is, for example, benzyl, 2-phenylethyl or 1-phenyleth-1-yl. (C₁₋₄ Alkyl)heteroaryl is, for example, pyridylmethyl or pyrimidinylmethyl. NHC(O)Heteroaryl is, for example, NHC(O)₂pyridyl. NHC(O)(C₁₋₄ Alkyl)phenyl is, for example, NHC(O)benzyl. NHC(O)₂(C₁₋₄ Alkyl)heteroaryl is, for example, NHC(O)CH₂pyridyl. NHS(O)₂Heteroaryl is, for example, NHS(O)₂pyridyl. NHS(b)₂(C₁₋₄ Alkyl)phenyl is, for example, NHS(O)₂benzyl. NHS(O)₂(C₁₋₄ Alkyl)heteroaryl is, for example, NHS(O)₂CH₂pyridyl. NHC(O)NHheteroaryl is, for example, NHC(O)NHpyridyl. NHC(O)NH(C₁₋₄ Alkyl)phenyl is, for example, NHC(O)NHbenzyl. NHC(O)NH(C₁₋₄ Alkyl)heteroaryl is, for example, NHC(O)NHCH₂pyridyl.

In one aspect of the invention k, m and n are, independently, 0 or 2. In a further aspect of the invention k, m and n are all 2.

In another aspect of the invention R¹ is phenyl {para-substituted by: halo, S(O)ₖ(C₁₋₆ alkyl), S(O)₂NH₂, S(O)₂NH(C₁₋₆ alkyl), S(O)₂N(C₁₋₆ alkyl)₂, cyano, NH₂, NH(C₁₋₆ alkyl), N(C₁₋₆ alkyl)₂, CO₂(C₁₋₆ alkyl), NHC(O)(C₁₋₆ alkyl), NHC(O)O(C₁₋₆ alkyl), NHS(O)₂(C₁₋₄ alkyl), NHC(O)phenyl, NHC(O)heteroaryl, NHC(O)(C₁₋₄ alkyl)phenyl, NHC(O)(C₁₋₄ alkyl)heteroaryl, NHS(O)₂phenyl, NHS(O)₂heteroaryl, NHS(O)₂(C₁₋₄ alkyl)phenyl, NHS(O)₂(C₁₋₄ alkyl)heteroaryl, NHC(O)NH(C₁₋₆ alkyl), NHC(O)NH(C₃₋₇ cycloalkyl), NHC(O)NHphenyl, NHC(O)NHheteroaryl, NHC(O)NH(C₁₋₄ alkyl)phenyl or NHC(O)NH(C₁₋₄ alkyl)heteroaryl; wherein the foregoing phenyl and heteroaryl groups are optionally substituted by halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃, [especially optionally substituted by halo]}. The variable k is 2.

In a further aspect of the invention R¹ is phenyl {para-substituted by: halo, cyano, CO₂(C₁₋₆ alkyl), NHC(O)(C₁₋₆ alkyl), NHS(O)₂(C₁₋₆ alkyl), NHC(O)phenyl, NHC(O)heteroaryl, NHC(O)(C₁₋₄ alkyl)phenyl, NHC(O)(C₁₋₄ alkyl)heteroaryl, NHS(O)₂phenyl, NHS(O)₂heteroaryl, NHS(O)₂(C₁₋₄ alkyl)phenyl or NHS(O)₂(C₁₋₄ alkyl)heteroaryl; wherein the foregoing phenyl and heteroaryl groups are optionally substituted by halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alky), NHC(O)(C₁₋₄ alkyl), NHS(O)₂(C₁₋₄ alkyl), C(O)(C₁₋₄ alkyl), CF₃ or OCF₃, [especially optionally substituted by halo]}. The variable k is 2.

In a still further aspect of the invention R¹ is phenyl {para-substituted by: halo, cyano, CO₂(C₁₋₆ alkyl), NHC(O)(C₁₋₆ alkyl), NHS(O)₂(C₁₋₆ alkyl), NHC(O)(C₁₋₄ alkyl)phenyl, NHC(O)(C₁₋₄ alkyl)heteroaryl, NHS(O)₂(C₁₋₄ alkyl)phenyl or NHS(O)₂(C₁₋₄ alkyl)heteroaryl; wherein the foregoing phenyl and heteroaryl groups are optionally substituted by halo}.

In another aspect R¹ is phenyl para-substituted by S(O)ₖ(C₁₋₄ alkyl), wherein k is 0, 1 or 2, (for example SCH₃, S(O)CH₃ or S(O)₂CH₃), NHS(O)₂(C₁₋₄ alkyl) (for example NHS(O)₂CH₃) or NHC(O)(C₁₋₄ alkyl) (for example NHC(O)CH₃). In yet another aspect R¹ is phenyl para-substituted by S(O)₂(C₁₋₄ alkyl) (for example S(O)₂CH₃), NHS(O)₂(C₁₋₄ alkyl) (for example NHS(O)₂CH₃) or NHC(O)(C₁₋₄ alkyl) (for example NHC(O)CH₃). In a still further aspect R¹ is phenyl para-substituted by S(O)₂(C₁₋₄ alkyl) (for example S(O)₂CH₃).

In a further aspect of the invention R² is phenyl or heteroaryl, either of which is optionally substituted in the ortho or meta position (that is ortho or meta to the point of attachment of the R² ring to the the rest of the structure of formula (I)) by halo, C₁₋₄ alkyl, C₁₋₄ alkoxy, S(O)ₙ(C₁₋₄ alkyl), nitro, cyano or CF₃.

In another aspect of the invention R² is phenyl or heteroaryl, either of which is optionally substituted in the ortho or meta position (that is ortho or meta relative to the position of attachment of that ring to the structure of formula (I)) by halo, C₁₋₄ alkyl, C₁₋₄ alkoxy, S(O)ₙ(C₁₋₄ alkyl), nitro, cyano or CF₃; wherein n is 0, 1 or 2, for example 0 or 2.

In yet another aspect R² is optionally substituted phenyl (especially optionally substituted by halo (such as chloro or fluoro), cyano, methyl, ethyl, methoxy, ethoxy or CF₃). In one aspect said substitution is on the ortho or meta position of the phenyl ring.

In a further aspect R² is optionally substituted phenyl (especially optionally substituted by halo or CF₃). For example R² is 3-fluorophenyl, 3-chlorophenyl, 4-fluorophenyl or 4-CF₃-phenyl.

In a still further aspect R² is phenyl, mono-fluorophenyl (for example 3-fluorophenyl or 4-fluorophenyl), difluorophenyl (for example 3,4-difluorophenyl or 3,5-dofluorophenyl), mono-chlorophenyl (for example 3-chlorophenyl) or mono-(C₁₋₄ alkoxy)phenyl (for example 4-methoxyphenyl). In a still further aspect R² is phenyl or mono-fluorophenyl (for example 3-fluorophenyl or 4-fluorophenyl).

In another aspect of the invention R³ is hydrogen or methyl. When R³ is C₁₋₄ alkyl (such as methyl) the carbon to which R³ is attached has, for example, the R absolute configuration. In yet another aspect of the invention R³ is hydrogen. In a further aspect of the invention R³ is methyl.

In a still further aspect of the invention R⁴ is ethyl.

In another aspect of the invention R⁵ is hydrogen, halo, hydroxy, nitro, cyano, C₁₋₄ alkyl, C₁₋₄ alkoxy, CF₃ or OCF₃. In a further aspect R⁵ is hydrogen.

In a still further aspect of the invention R⁶ is methyl or ethyl (such as methyl). A compound of the invention wherein R⁶ is methyl. A compound of the invention wherein the S(O)₂R⁶ group of formula (I) is para disposed to the remainer of the structure of formula (1), that is, it is as shown here:

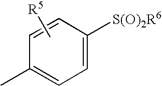

In another aspect of the invention R⁶ is C₁₋₄ alkyl and wherein the S(O)₂R⁶ group of formula (I) is para disposed to the remainder of the structure of formula (I).

In a still further aspect the present invention provides a compound of formula (Ia): 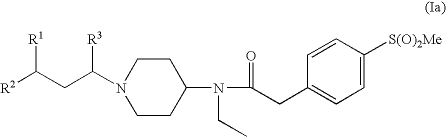
wherein R¹, R² and R³ are as defined above; provided that:
when R³ is hydrogen and R² is unsubstituted phenyl then R¹ is not para-methoxy-phenyl, para-methyl-phenyl or para-trifluoromethyl-phenyl;
when R³ is hydrogen and R² is unsubstituted phenyl, pyrid-2-yl or pyrid-4-yl then R¹ is not para-chloro-phenyl;
when R³ is hydrogen and R² is unsubstituted phenyl then R¹ is not 3,4-dichlorophenyl; and,
when R³ is hydrogen and R² is meta-chloro-phenyl, unsubstituted phenyl or thiophen-3-yl then R¹ is not para-fluoro-phenyl.

The present invention further provides a compound of formula (I) or (Ia) wherein R¹ is phenyl para-substituted by S(O)₂(C₁₋₄ alkyl) (such as S(O)₂CH₃); R² is phenyl or mono-fluorophenyl (such as 3-fluorophenyl); and R³ is hydrogen or C₁₋₄ alkyl (such as methyl) (R³ is, for example, hydrogen); said compound being in free base form or in the form of a hydrochloride adduct.

The following compounds illustrate the invention.

TABLE I
Table I comprises compounds of formula (Ia).

| | |
|---|---|
| | (Ia) |
| 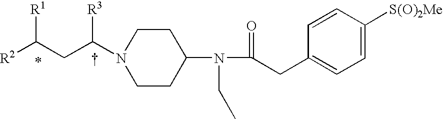 | |

| Compound | | | | | | | LCMS |
|---|---|---|---|---|---|---|---|
| No. | R¹ | R² | R³ | Adduct | *Chirality | ^{†}Chirality | (MH+) |
| 1 | 4-Chlorophenyl | Pyrid-3-yl | H | | | | 554 |
| 2 | 4-Chlorophenyl | Phenyl | H | | (−)isomer^{#} | | 553 |
| 3 | 4-Cyanophenyl | Phenyl | H | | | | 544 |
| 4 | 4-Methoxycarbonylphenyl | Phenyl | H | | | | 577 |
| 5 | 4-(Morpholin-4-ylcarbonyl)phenyl | Phenyl | H | | | | 632 |
| 6 | 4-Carboxamidophenyl | Phenyl | H | | | | 562 |
| 7 | 4-iso-propoxycarbonylphenyl | Phenyl | H | | | | 605 |
| 8 | 4-Fluorophenyl | Phenyl | H | | | | |
| 9 | 4-Aminophenyl | Phenyl | H | | | | 534 |
| 10 | 4-tert-butyloxycarbonylaminophenyl | Phenyl | H | | | | 634 |
| 11 | 4-(Pyridin-2-ylacetylamino)phenyl | Phenyl | H | | | | 653 |
| 12 | 4-(Pyridin-3-ylacetylamino)phenyl | Phenyl | H | | | | 653 |
| 13 | 4-(Pyridin-4-ylacetylamino)phenyl | Phenyl | H | | | | 653 |
| 14 | 4-Phenylacetylaminophenyl | Phenyl | H | | | | 652 |
| 15 | 4-Butyrylaminophenyl | Phenyl | H | | | | 604 |
| 16 | 4-Acetylaminophenyl | Phenyl | H | | | | 576 |
| 17 | 4-Cyclohexylureidophenyl | Phenyl | H | | | | 659 |
| 18 | 4-Phenylureidophenyl | Phenyl | H | | | | 653 |
| 19 | 4-Benzoylaminophenyl | Phenyl | H | | | | 638 |
| 20 | 4-(4-Chlorobenzoylamino)phenyl | Phenyl | H | | | | 672 |
| 21 | 4-(2,2- | Phenyl | H | | | | 618 |
| | Dimethylpropionylamino)phenyl | | | | | | |
| 22 | 4-Phenylmethanesulfonylamino- | Phenyl | H | | | | 688 |
| | phenyl | | | | | | |
| 23 | 4-Ethanesulfonylaminophenyl | Phenyl | H | | | | 626 |
| 24 | 4-Methanesulfonylaminophenyl | Phenyl | H | | | | 612 |
| 25 | 4-Phenylphenyl | Phenyl | H | | | | 595 |
| 26 | 4-Chlorophenyl | 3-Fluorophenyl | H | | | | 571 |
| 27 | 4-Chlorophenyl | 3-Fluorophenyl | Methyl | | | R isomer | 585 |
| 28 | 4-Benzenesulfonylaminophenyl | Phenyl | H | | | | 675 |
| 29 | 4-iso-propylsulfonylaminophenyl | Phenyl | H | | | | |
| 30 | 4-Cyanophenyl | Phenyl | Methyl | | | R isomer | |
| 31 | 4-Cyanophenyl | 3-Fluorophenyl | Methyl | | | R isomer | |
| 32 | 4-Methanesulfonylaminophenyl | Phenyl | Methyl | | | R isomer | |
| 33 | 4-Methanesulfonylaminophenyl | 3-Fluorophenyl | Methyl | | | R isomer | |
| 34 | 4-Acetylaminophenyl | Phenyl | Methyl | | | R isomer | |
| 35 | 4-Acetylaminophenyl | 3-Fluorophenyl | Methyl | | | R isomer | |
| 36 | 4-Chlorophenyl | Thien-2-yl | H | | | | |
| 37 | 4-Cyanophenyl | Thien-2-yl | H | | | | |
| 38 | 4-Chlorophenyl | Thiazol-4-yl | H | | | | |
| 39 | 4-Cyanophenyl | Thiazol-4-yl | H | | | | |
| 40 | 4-Methanesulfonylphenyl | Phenyl | H | | | | 597 |
| 41 | 4-Methanethiophenyl | Phenyl | H | | | | 565 |
| 42 | 4-iso-propylaminocarboxy- | Phenyl | H | | | | 619 |
| | aminophenyl | | | | | | |
| 43 | 4-tert-butoxycarbonylaminophenyl | 3-Fluorophenyl | H | | | | 652 |
| 44 | 4-Aminophenyl | 3-Fluorophenyl | H | hydrochloride | | | 552 |
| 45 | 4-Acetylaminophenyl | 3-Fluorophenyl | H | | | | 594 |
| 46 | 4-Methanesulfonylaminophenyl | 3-Fluorophenyl | H | | | | 630 |
| 47 | 4-(4-Methanesulfonylbenzoylamino)- | 3-Fluorophenyl | H | hydrochloride | | | 734 |
| | phenyl | | | | | | |
| 48 | 4-(5-Methanesulfonylthien-2-yl- | 3-Fluorophenyl | H | hydrochloride | | | 740 |
| | acetylamino)phenyl | | | | | | |
| 49 | 4-Methanesulfonylammophenyl | 3-Fluorophenyl | H | hydrochloride | (+)isomer^{†} | | 630 |
| 50 | 4-Methanesulfonylphenyl | Phenyl | H | hydrochloride | S isomer | | 597 |
| 51 | 4-Methanesulfonylphenyl | 3-Fluorophenyl | H | hydrochloride | R isomer | | 615 |
| 52 | 4-Methanesulfonylphenyl | 4-Fluorophenyl | H | hydrochloride | S isomer | | 615 |
| 53 | 4-Methanesulfonylphenyl | 3-Chlorophenyl | H | hydrochloride | R isomer | | 631 |
| 54 | 4-Methanesulfonylphenyl | 3,4-Difluorophenyl | H | hydrochloride | R isomer | | 633 |
| 55 | 4-Methanesulfonylphenyl | 4-Methoxyphenyl | H | hydrochloride | S isomer | | 627 |
| 56 | 4-Methanesulfonylphenyl | 3,5-Difluorophenyl | H | hydrochloride | R isomer | | 633 |
| 57 | 4-Methanesulfonylphenyl | Phenyl | Methyl | | S isomer | R isomer | |
| 58 | 4-Methanesulfonylphenyl | 4-Fluorophenyl | Methyl | | S isomer | R isomer | |
| 59 | 4-Methanesulfonylphenyl | 3,4-Difluorophenyl | Methyl | | R isomer | R isomer | |
| 60 | 4-Methanesulfonylphenyl | 3,5-Difluorophenyl | Methyl | | R isomer | R isomer | |
| 61 | 4-Methanesulfonylphenyl | 3-Fluorophenyl | Methyl | | R isomer | R isomer | |
| 62 | 4-Methanesulfonylphenyl | 3-Trifluoromethylphenyl | Methyl | | R isomer | R isomer | |
| 63 | 4-Methanesulfonylphenyl | 3-Trifluoromethylphenyl | H | | R isomer | | |
| 64 | 4-Aminophenyl | 3-Fluorophenyl | H | | | | |
| 65 | 4-(4-Methanesulfonylbenzoylamino) | 3-Fluorophenyl | H | | | | |
| | phenyl | | | | | | |
| 66 | 4-(5-Methanesulfonylthien-2-yl- | 3-Fluorophenyl | H | | | | |
| | acetylamino)phenyl | | | | | | |
| 67 | 4-Methanesulfonylaminophenyl | 3-Fluorophenyl | H | | (+)isomer^{†} | | |
| 68 | 4-Methanesulfonylphenyl | Phenyl | H | | S isomer | | |
| 69 | 4-Methanesulfonylphenyl | 3-Fluorophenyl | H | | R isomer | | |
| 70 | 4-Methanesulfonylphenyl | 4-Fluorophenyl | H | | S isomer | | |
| 71 | 4-Methanesulfonylphenyl | 3-Chlorophenyl | H | | R isomer | | |
| 72 | 4-Methanesulfonylphenyl | 3,4-Difluorophenyl | H | | R isomer | | |
| 73 | 4-Methanesulfonylphenyl | 4-Methoxyphenyl | H | | S isomer | | |
| 74 | 4-Methanesulfonyiphenyl | 3,5-Difluorophenyl | H | | R isomer | | |
| 75 | 4-Methanesulfinylpheny | Phenyl | H | | | | |

^{#}Compound separated from racemate using 10 micron Chiralcel OJ (250 mm × 20 mm). As eluent comprises formic acid compound from column is formic acid adduct which was treated with base to yield free base, Compound No. 2. Estimated αD −7.29(CHCl₃, 589 nm, c = 0.425)
^{†}Compound separated from racemate using 10 micron Chiralcel OJ (250 mm × 20 mm). As eluent comprises formic acid compound from column is formic acid adduct which was treated with base to yield free base, Compound 67. Compound No. 67 used to form Compound 49. Estimated αD +5.85(CHCl₃, 589 nm, c = 2.00) for Compound No. 49.

In another aspect the present invention provides each individual compound recited in Table I. In yet another aspect the present invention provides Compound No. 2 of Table L or a pharmaceutically acceptable salt thereof or a solvate thereof; Compound No. 50, 51, 67 or 68 of Table I, or a pharmaceutically acceptable salt thereof or a solvate thereof.

The compounds of the invention can be prepared as shown in the processes on pages marked Schemes 2 to 4 below, while Scheme 1 shows the preparation of an intermediate used in Schemes 2 and 3. (In Schemes 1 to 4 PG is a protecting Group; Ac is acetyl; Bn is benzyl, Bz is benzoyl; LDA is lithium diisopropylamide; and TMEDA is N,N,N′,N′-tetramethylethyenediamine. Suitable coupling agents include PyBrOP or HATU.)

A compound of the invention can be prepared by reductive amination of a compound of formula (II or (IIa): 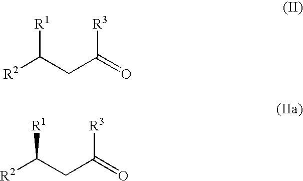
with a compound of formula (III): 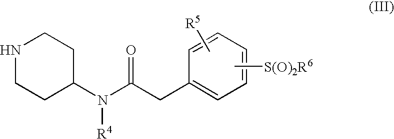
in the presence of NaBH(OAc)₃ (wherein Ac is C(O)CH₃) and acetic acid, in a suitable solvent (such as a C₁₋₆ aliphatic alcohol, for example ethanol) at room temperature (for example 10-30° C.)

Alternatively, a compound of the invention can be prepared by coupling a compound of formula (IV) or (IVa): 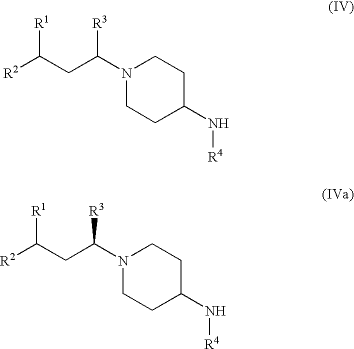
with a compound of formula (V): 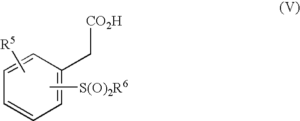
in the presence of a suitable coupling agent (for example PyBrOP or HATU) in the presence of a suitable base (such as a tertiary amine, for example diisopropylethylamine) in a suitable solvent (for example N-methylpyrrolidinone or a chlorinated solvent, such as dichloromethane) at room temperature (for example 10-30° C.).

The starting materials for these preparative methods and Schemes are either commercially available or can be prepared by literature methods, adapting literature methods or by following or adapting Methods herein described.

In a further aspect the invention provides processes for preparing the compounds of the invention. Many of the intermediates in the processes are novel and these are provided as further features of the invention.

The compounds of the invention have activity as pharmaceuticals, in particular as modulators (such as agonists, partial agonists, inverse agonists or antagonists) of chemokine receptor (especially CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

The compounds of the present invention are also of value in inhibiting the entry of viruses (such as human immunodeficiency virus (HIV)) into target calls and, therefore, are of value in the prevention of infection by viruses (such as HIV), the treatment of infection by viruses (such as HIV) and the prevention and/or treatment of acquired immune deficiency syndrome (AIDS).

According to a further feature of the invention there is provided a compound of the invention, or a pharmaceutically acceptable salt thereof or a solvate thereof, for use in a method of treatment of a warm blooded animal (such as man) by therapy (including prophylaxis).

According to a further feature of the present invention there is provided a method for modulating chemokine receptor activity (especially CCR5 receptor activity) in a warm blooded animal, such as man, in need of such treatment, which comprises administering to said animal an effective amount of a compound of the present invention, or a pharmaceutically acceptable salt thereof or a solvate thereof.

The present invention also provides the use of a compound of the invention, or a pharmaceutically acceptable salt thereof or a solvate thereof, as a medicament, especially a medicament for the treatment of transplant rejection, respiratory disease, psoriasis or rheumatoid arthritis (especially rheumatoid arthritis). [Respiratory disease is, for example, COPD, asthma {such as bronchial, allergic, intrinsic, extrinsic or dust asthma, particularly chronic or inveterate asthma (for example late asthma or airways hyper-responsiveness)} or rhinitis {acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis}; and is particularly asthma or rhinitis].

In another aspect the present invention provides the use of a compound of the invention, or a pharmaceutically acceptable salt thereof or a solvate thereof, in the manufacture of a medicament for use in therapy (for example modulating chemokine receptor activity (especially CCR5 receptor activity (especially rheumatoid arthritis)) in a warm blooded animal, such as man).

The invention also provides a compound of the invention, or a pharmaceutically acceptable salt thereof or a solvate thereof, for use as a medicament, especially a medicament for the treatment of rheumatoid arthritis.

In another aspect the present invention provides the use of a compound of the invention, or a pharmaceutically acceptable salt thereof or a solvate thereof, in the manufacture of a medicament for use in therapy (for example modulating chemokine receptor activity (especially CCR5 receptor activity (especially rheumatoid arthritis)) in a warm blooded animal, such as man).

The invention further provides the use of a compound of the invention, or a pharmaceutically acceptable salt thereof, in the manufacture of a medicament for use in the treatment of:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;
(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;
(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia greata or vernal conjunctivitis;
   (4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
   (5) (Allograft rejection) acute and chronic following, for example, transplantation of kidney, heart, liver, lung, bone marrow, skin or cornea; or chronic graft versus host disease; and/or
   (6) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura or disorders of the menstrual cycle; in a warm blooded animal, such as man.

The present invention further provides a method of treating a chemokine mediated disease state (especially a CCR5 mediated disease state) in a warm blooded animal, such as man, which comprises administering to a mammal in need of such treatment an effective amount of a compound of the invention, or a pharmaceutically acceptable salt thereof or solvate thereof.

In order to use a compound of the invention, or a pharmaceutically acceptable salt thereof or solvate thereof, for the therapeutic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises a compound of the invention, or a pharmaceutically acceptable salt thereof or a solvate thereof (active ingredient), and a pharmaceutically acceptable adjuvant, diluent or carrier. In a further aspect the present invention provides a process for the preparation of said composition which comprises mixing active ingredient with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of active ingredient.

In another aspect a pharmaceutical composition of the invention is one suitable for intravenous, subcutaneous or intramuscular injection.

Each patient may receive, for example, an intravenous, subcutaneous or intramuscular dose of 0.01 mgkg¹ to 100 mgkg⁻¹ of the compound, preferably in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the compound of the invention, or a pharmaceutically acceptable salt thereof or a solvent thereof (hereafter Compound X), for therapeutic or prophylactic use in humans:

(a)

| Tablet I | mg/tablet |
|---|---|
| Compound X | 100 |
| Lactose Ph.Eur. | 179 |
| Croscarmellose sodium | 12.0 |
| Polyvinylpyrrolidone | 6 |
| Magnesium stearate | 3.0 |

(b)

| Tablet II | mg/tablet |
|---|---|
| Compound X | 50 |
| Lactose Ph.Eur. | 229 |
| Croscarmellose sodium | 12.0 |
| Polyvinylpyrrolidone | 6 |
| Magnesium stearate | 3.0 |

(c)

| Tablet III | mg/tablet |
|---|---|
| Compound X | 1.0 |
| Lactose Ph.Eur. | 92 |
| Croscarmellose sodium | 4.0 |
| Polyvinylpyrrolidone | 2.0 |
| Magnesium stearate | 1.0 |

(d)

| Capsule | mg/capsule |
|---|---|
| Compound X | 10 |
| Lactose Ph.Eur. | 389 |
| Croscarmellose sodium | 100 |
| Magnesium stearate | 1.0 |

(e)

| Injection I | (50 mg/ml) |
|---|---|
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. The tablets (a)-(c) may be enteric coated by conventional means, for example to provide a coating of cellulose acetate phthalate.

The invention will now be illustrated by the following non-limiting Examples in which, unless stated otherwise:
(i) temperatures are given in degrees Celsius (° C.); operations were carried out at room or ambient temperature, that is, at a temperature in the range of 18-25° C.;
(ii) organic solutions were dried over anhydrous magnesium sulfate; evaporation of solvent was carried out using a rotary evaporator under reduced pressure (600-4000 Pascals; 4.5-30 mm Hg) with a bath temperature of up to 60° C.;
(iii) chromatography unless otherwise stated means flash chromatography on silica gel; thin layer chromatography (TIC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology ltd., 1st House, Duffryn Industial Estate, Ystrad Mynach, Hengoed, Mid Glamorgan, UK. Where “Argonaut™ PS-tris-anine scavenger resin” is referred to, this means a tris-2-aminoethyl)amine polystyrene resin obtained from Argonaut Technologies Inc., 887 Industrial Road, Suite G, San Carlos, Cali., USA.
(iv) in general, the course of reactions was followed by TLC and reaction times are given for illustration only;
(v) yields, when given, are for illustration only and are not necessarily those which can be obtained by diligent process development; preparations were repeated if more material was required;
(vi) when given, ¹H NMR data is quoted and is in the form of delta values for major diagnostic protons, given in parts per million (ppm) relative to tetramethylsilane (TMS) as an internal standard, determined at 300 MHz using perdeuterio DMSO (CD₃SOCD₃) as the solvent unless otherwise stated; coupling constants (J) are given in Hz;
(vii) chemical symbols have their usual meanings; SI units and symbols are used;
(viii) solvent ratios are given in percentage by volume;
(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion —CM+H)⁺;
(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺ and

(xi) the following abbreviations are used:

| | |
|---|---|
| DMSO | dimethyl sulfoxide; |
| DMF | N-dimethylformamide; |
| DCM | dichloromethane; |
| THF | tetrahydrofuran; |
| DIPEA | N,N-diisopropylethylamine; |
| NMP | N-methylpyrrolidinone; |
| HATU | O-(7-Azabenzotriazol-1-yl)-N,N,N′,N′-tetramethyluronium |
| | hexafluorophosphate; |
| HBTU | O-(7-Benzotriazol-1-yl)-N,N,N′,N′-tetramethyluronium |
| | hexafluorophosphate; |
| Boc | tert-butoxycarbonyl |
| MeOH | methanol; |
| EtOH | ethanol; and |
| EtOAc | ethyl acetate. |

### EXAMPLE 1

This Example illustrates the preparation of N-[1-(3-phenyl-3-[4-phenylphenyl]propyl)-piperidin-4-yl-N-ethyl-4-methanesulfonylphenylacetamide (Compound No. 25 of Table I).

To a mixture of N-(4-piperidinyl)-N-ethyl-4-methanesulfonylphenylacetamide (Method A; 500 mg, 1.54 mmol) and 3-phenyl-3-(4-phenylphenyl)propionaldehyde (Method B; 449 mg, 1.54 mmol) in DCM (10 mL) and ethanol (2 mL) was added one drop of acetic acid and the resulting mixture stirred at room temperature for 10 min. Sodium triacetoxyborohydride (327 mg, 1.54 mmol) was added and the resulting mixture was stirred at room temperature for 2 h. The reaction mixture was washed with 2M aqueous sodium hydroxide (2×10 mL) and eluted through a 10 g SCX cartridge with DCM (2×10 mL), methanol (2×10 mL) and finally 0.5M ammonia in methanol (3×10 mL) to yield crude product which was purified by silica gel chromatography (eluent: DCM then ethyl acetate then 10% methanol in ethyl acetate) to yield the title compound (406 mg); NMR: 0.95-1.3 (m, 3H) 1.3-1.95 (m, 8H) 2.2 (m, 3H) 2.8 (m, 2H) 3.15 (s, 3H) 3.8 (m, 2H) 4.05 (m, 3H) 7.05-7.6 (m, 16H) 7.8 (d, 2H); MS: 595.

The procedure described in Example 1 can be repeated using different aldehydes (such as 3-phenyl-3-(pyridin-2-yl)propionaldehyde (Method G), 3-(4-chlorophenyl)-3-(3-fluorophenyl)propionaldehyde (Method I), (S)-3-phenyl-3-(4-methanesulfonylphenyl)propionaldehyde (Method N), (R)-3-(3-fluorophenyl)-3-(4-methanesulfonylphenyl)propionaldehdye (Method 0), (S)-3-(4-fluorophenyl)-3-(4-methanesulfonylphenyl)propionaldehdye (Method P), (R)-3-(3-chlorophenyl)-3-(4-methanesulfonylphenyl)propionaldehdye (Method Q), (R)-3-(3,4-difluorophenyl)-3-(4-methanesulfonylphenyl)propionaldehdye (Method R), (S)-3-(4-methoxyphenyl)-3-(4-methanesulfonylphenyl)propionaldehdye (Method S), or (R)-3-(3,5-difluorophenyl)-3-(4-methanesulfonylphenyl)propionaldehdye (Method T)) in place of 3-phenyl-3-(4-phenylphenyl)propionaldehyde.

### EXAMPLE 2

This Example illustrates the preparation of N-[1-(3-phenyl-3-[4-methoxycarbonyl-phenyl]propyl)-piperidin-4-yl)-N-ethyl-4-methanesulfonylphenylacetamide (Compound No. 4 of Table I).

This was prepared from 3-phenyl-3-(4-methoxycarbonylphenyl)propionaldehyde (Method F) and N-(4-piperidinyl)-N-ethyl-4-methanesulfonylphenylacetamide using a method similar to that used to prepare N-[1-(3-phenyl-3-[4-phenylphenyl]propyl)-piperidin-4-yl]-N-ethyl-4-methanesulfonylphenylacetamide (Example 1). NMR (CDCl₃): 1.1 and 1.2 (t; 3H), 1.5 (m, 1H), 1.8 (m, 2H), 2.0 (br t, 2H), 2.2 (m, 4H), 2.9 (m, 2H), 3.0 (s, 3H), 3.3 (m, 2H), 3.8 (m, 2H), 3.9 (s, 3H), 4.1 (m, 2H), 4.4 (m, 11), 7.2 (m, 7H), 7.5 (m, 2H) and 7.9 (m, 4H); MS: 577.

### EXAMPLE 3

This Example illustrates the preparation of N-[1-(3-phenyl-3-[4-{morpholin-4-ylcarbonyl}phenyl]propyl)-piperidin-4-yl]-N-ethyl-4-methanesulfonylphenylacetamide (Compound No. 5 of Table I).

To a solution of N-[1-(3-phenyl-3-[4-carboxyphenyl]propyl)-piperidin-4-yl]-N-ethyl-4-methanesulfonylphenylacetamide hydrochloride (Method H; 100 mg, 0.16 mmol) in DCM (4 mL) was added oxalyl chloride (0.05 mL) and the resulting mixture was stirred at room temperature for 3 h. The mixture was cooled to 0° C. and a solution of morpholine in DCM was added dropwise until a pH of 9 was achieved. The reaction mixture was washed with water and brine, dried (MgSO₄) and evaporated. The residue was purified by silica gel chromatography (eluent: 2% ammonia/10% methanol in DCM) giving the title compound (34 mg); NMR (CDCl₃): 1.1 and 1.2 (t, 3H), 1.4 (m, 1H), 1.6 (m, 21), 1.8 (m, 2H), 2.0 (m, 2H), 2.2 (m, 4H), 2.9 (m, 2H), 3.0 (s, 3H), 3.3 (q, 2H), 3.6 (br m, 7H), 3.8 (m, 2H), 4.0 (m, 1H), 4.4 (m, 1H), 7.2 (m, 9H), 7.5 (m, 2H) and 7.9 (d, 2H); MS: 632.

### EXAMPLE 4

This Example illustrates the preparation of N-[1-(3-phenyl-3-[4-carboxamidophenyl]-3-propyl)-piperidin-4-yl]-N-ethyl-4-methanesulfonylphenylacetamide (Compound No. 6 of Table I).

To a solution of N-[1-(3-phenyl-3-[4-carboxyphenyl]propyl)-piperidin-4-yl]-N-ethyl-4-methanesulfonylphenylacetamide hydrochloride (150 mg, 0.25 mmol) in DCM (4 mL) was added oxalyl chloride (0.022 mL, 0.25 mmol) and the resulting mixture was stirred at room temperature for 18 h. A solution of ammonia in methanol (10 mL) was added and the resulting mixture was stirred at room temperature for 2 h. The reaction mixture was washed with water and evaporated. The residue was purified by silica gel chromatography (eluent: 1% ammonia/10% methanol in DCM) giving the title compound (23 mg); NMR (CDCl₃): 1.1 and 1.2 (t, 3H), 1.5 (m, 2H), 1.6 (m, 1H), 1.8 (m, 2H), 2.0 (m, 2H), 2.2 (m, 4H), 2.9 (m, 2H), 3.0 (s, 3M), 3.3 (q, 2H), 3.8 (m, 2H), 4.0 (m, 2H), 4.4 (m, 1H), 7.2 (in, 7H), 7.4 (m, 2H), 7.7 (m, 2H) and 7.9. (d, 2H); MS: 562.

### EXAMPLE 5

This Example illustrates the preparation of N-[1-(3-phenyl-3-[4-isopropoxycarbonyl-phenyl]propyl)-piperidin-4-yl]-N-ethyl-4-methanesulfonylphenylacetamide hydrochloride (Compound No. 7 of Table I).

To a suspension of N-(1-(3-phenyl-3-[4-carboxyphenyl]propyl)-piperidin-4-yl]-N-ethyl-4-methanesulfonylphenylacetamide hydrochloride (40 mg, 0.07 mmol) in 2-propanol (4 mL) was added thionyl chloride (3 drops). The resulting mixture was heated to reflux for 18 h, allowed to cool and evaporated. The residue was triturated with diethyl ether to give the title compound (31 mg); NMR: 1.0 and 1.1 (t, 3H), 1.3 (t, 3H), 1.7 (m, 2H), 2.2 (m, 2H), 2.8 (m, 2M), 3.0 (m, 2H), 3.2 (s, 3H), 3.3 (m, 4H), 3.5 (m, 2H), 3.8 (m, 2H), 4.0 and 4.2 (m, 1H), 4.1 (m, 1H), 7.2 (m, 5H), 7.5 (br d, 4H) and 7.8 (br t, 4H); MS: 605.

### EXAMPLE 6

This Example illustrates the preparation of N-[1-(3-phenyl-3-[4-cyanophenyl]propyl)-piperidin-4-yl]-N-ethyl-4-methanesulfonylphenylacetamide (Compound No. 3 of Table I).

To a mixture of N-[1-(3-phenyl-3-[4-carboxamidophenyl]propyl)-piperidin-4-yl]-N-ethyl-4-methanesulfonylphenylacetamide (Example 4; 0.33 mmol) in dioxane (5 mL) and pyridine (0.05 mL, 0.6 mmol) at 0° C. was added trifluoroacetic anhydride (0.1 mL, 0.66 mmol) and the resulting mixture stirred at room temperature for 1 h. The reaction mixture was washed with water and brine, dried (MgSO₄) and evaporated. The residue was purified by eluting through an SCX cartridge with methanol then 2M ammonia in methanol to yield the title compound (27 mg); NMR (CDCl₃): 1.1 and 1.3 (t, 3H), 1.5 (m, 1H), 1.7 (m, 2H), 1.8 (m, 2H), 2.0 (m, 1H), 2.2 (m, 4H), 2.9 (m, 2H), 3.0 (s, 3H), 3.3 (q, 2H), 3.8 (m, 2H), 4.0 (m, 11H), 4.4 (m, 1H), 7.2 (m, 7H), 7.4 (m, 2H), 7.6 (m, 2H) and 7.9 (d, 21); MS: 544.

### EXAMPLE 7

This Example illustrates the preparation of N-[1-(3-phenyl-3-[4-aminophenyl]propyl)-piperidin-4-yl]-N-ethyl-4-methanesulfonylphenylacetamide (Compound No. 9 of Table I).

N-[1-(3-Phenyl-3-[4-Boc-aminophenyl]propyl)-piperidin-4-yl]-N-ethyl-4-methanesulfonylphenylacetamide (Example 8; 6 g, 9.5 mmol) was dissolved in trifluoroacetic acid (25 mL) and the resulting mixture was stirred at room temperature for 2 h. The mixture was evaporated and the residue dissolved in 2M aqueous sodium hydroxide (50 mL) and DCM (50 mL). The aqueous phase was extracted with DCM (3×25 mL) and the combined organic phases dried (MgSO₄) then eluted through a 50 g SCX cartridge with DCM (3×25 mL), methanol (3×25 mL) and 1M ammonia in methanol (5×25 mL) to give the title compound (4.5 g); MS: 534.

### EXAMPLE 8

This Example illustrates the preparation of N-[1-(3-phenyl-3-[4-Boc-aminophenyl]propyl)-piperidin-4-yl]-N-ethyl-4-methanesulfonylphenylacetamide (Compound No. 10 of Table I).

This was prepared from 3-phenyl-3-(4-Boc-aminophenyl)propionaldehyde (Method C) and N-(4-piperidinyl)-N-ethyl-4-methanesulfonylphenylacetamide using a method similar to that used to prepare N-[1-(3-phenyl-3-[4-phenylphenyl]propyl)-piperidin-4-yl]-N-ethyl-4-methanesulfonylphenylacetamide (Example 1).

### EXAMPLE 9

This Example illustrates the preparation of N-[1-(3-phenyl-3-(4-pyridin-2-ylacetylamino phenyl]propyl)-piperidin-4-yl]-N-ethyl-4-methanesulfonylphenylacetamide (Compound No. 11 of Table I).

To a solution of 2-pyridylacetic acid hydrochloride (81 mg, 4.7 mmol) in DCM (10 mL) was added triethylamine (47 mg, 4.7 mmol) and carbonyl diimidazole (75 mg, 4.7 mmol). The resulting mixture was stirred at room temperature for 4 h. N-[1-(3-Phenyl-3-[4-aminophenyl]propyl)-piperidin-4-yl]-N-ethyl-4-methanesulfonylphenylacetamide (250 mg, 4.7 mmol) was added and the resulting mixture was stirred at room temperature for 18 h. Isocyanate scavenger resin (0.3 g) was added and the resulting mixture was stirred at room temperature for 2 h before filtration and washing with 2M aqueous sodium hydroxide (10 mL). The filtrate was extracted with DCM and extracts dried and evaporated to give the title compound (184 mg); MS: 653.

The procedure described in Example 9 can be repeated using different carboxylic acids (such as 3-pyridylacetic acid hydrochloride or 4-pyridylacetic acid hydrochloride) in place of 2-pyridylacetic acid hydrochloride.

### EXAMPLE 10

This Example illustrates the preparation of N-[1-(3-phenyl-3-[4-phenylacetylamino phenyl]propyl)-piperidin-4-yl]-N-ethyl-4-methanesulfonylphenylacetamide (Compound No. 14 of Table I).

To a mixture of N-[1-(3-phenyl-3-[4-aminophenyl]propyl)-piperidin-4-yl]-N-ethyl-4-methanesulfonylphenylacetamide (250 mg, 4.7 mmol) and triethylamine (47 mg, 4.7 mmol) in DCM (10 mL) was added phenylacetyl chloride (72 mg, 4.7 mmol) and the resulting mixture was stirred at room temperature for 18 h. Trisamine scavenger resin (100 mg) was added and the resulting mixture was stirred at room temperature for 2 h before filtration. The filtrate was washed with saturated aqueous sodium bicarbonate solution (10 mL), dried and eluted through a 10 g SCX cartridge with DCM (4×10 mL), methanol (4×10 mL) and 1M ammonia in methanol (4×10 mL) to give the title compound (202 mg); MS: 652.

The procedure described in Example 10 can be repeated using different carbonyl chlorides (such as benzoyl chloride, 4-chlorobenzoyl chloride or pivaloyl chloride) or sulfonyl chlorides (such as benzene sulfonyl chloride or methane sulfonyl chloride) or isocyanates (such as phenyl isocyanate or cyclohexyl isocyanate) in place of phenylacetic acid, and different anilines (such as N-[1-(3-[3-fluorophenyl]-3-[4-aminophenyl]propyl)-piperidin-4-yl]-N-ethyl-4-methanesulfonylphenylacetamide) (Example 12) in place of N-[1-(3-phenyl-3-[4-aminophenyl]propyl)-piperidin-4-yl]-N-ethyl-4-methanesulfonylphenylacetamide. When isocyanates are employed the triethylamine is omitted from the reaction.

### EXAMPLE 11

This Example illustrates the preparation of (2R)-N-[1-(4-[4-chlorophenyl]-4-[3-fluorophenyl]but-2-yl)piperidin-4-yl]-N-ethyl-4-methanesulfonylphenylacetamide (Compound No. 27 of Table I).

To a mixture of (2R)-1-(4-[4-chlorophenyl]-4-[3-fluorophenyl]but-2-yl)-4-ethylaminopiperidine (Method K; 0.22 g, 0.56 mmol) and 4-methanesulfonylphenylacetic acid (0.33 g, 0.62 mmol) in DCM (10 mL) was added diisopropylcarbodiimide (0.1 mL, 0.62 mmol) and the resulting mixture was stirred at room temperature for 18 h before evaporation. The crude product was purified by eluting through a Bond Elut with DCM then 1% ammonia/10% methanol in DCM to give the title compound as a solid (0.36 g); NMR (CDCl₃): 0.9 (d, 3H), 1.2 (t, 3H), 1.5 (m, 1H), 1.6 (m, 2H), 1.8 (m, 1H), 2.0 (m, 1H), 2.2 (m, 2H), 2.4 (m, 1H), 2.5 (m, 1H), 2.7 (m, 1H), 3.0 (s, 3H), 3.3 (m, 2H), 3.8 (m, 2H), 3.9 and 4.3 (m, 1H), 4.2 (m, 1H), 4.4 (m, 1H), 6.9 (m, 3H), 7.2 (m, 5H), 7.5 (m, 2H) and 7.9 (d, 2H); MS: 585.

### EXAMPLE 12

This Example illustrates the preparation of N-[1-(3-[3-fluorophenyl]-3-[4-aminophenyl]propyl)-piperidin-4-yl]-N-ethyl-4-methanesulfonylphenylacetamide hydrochloride (Compound No. 44 of Table I).

This was prepared from N-[1-(3-[3-fluorophenyl]-3-[4-Boc-aminophenyl]propyl)-piperidin-4-yl]-N-ethyl-4-methanesulfonylphenylacetamide (Example 13) using a method similar to that used to prepare N-[1-(3-phenyl-3-[4-aminophenyl]propyl)-piperidin-4-yl]-N-ethyl-4-methanesulfonylphenylacetamide (Example 7); NMR: 1.0 and 1.15 (t, 3H), 1.7 (m, 2H), 2.3 (m, 2H), 2.8 (m, 2H), 2.5 (m, 2H), 3.0 (m, 2H), 3.2 (s, 3H), 3.2 and 3.3 (m, 2H), 3.5 (m, 2H), 3.7 and 4.1 (m, 1H), 3.8 and 3.9 (s, 2H), 4.35 (m, 1H), 7.0 (m, 1H), 7.2 (m, 2H), 7.35 (m, 3H), 7.45 (m, 4H), 7.8 (d, 2H); MS: 552 (MH+).

### EXAMPLE 13

This Example illustrates the preparation of N-[1-(3-[3-fluorophenyl]-3-[4-Boc-aminophenyl]propyl)-piperidin-4-yl]-N-ethyl-4-methanesulfonylphenylacetamide (Compound No. 43 of Table I).

This was prepared from 3-(3-fluorophenyl)-3-(4-Boc-aminophenyl)propionaldehyde (Method L) and N-(4-piperidinyl)-N-ethyl-4-methanesulfonylphenylacetamide using a method similar to that used to prepare N-[1-(3-phenyl-3-[4-phenylphenyl]propyl)-piperidin-4-yl]-N-ethyl-4-methanesulfonylphenylacetamide (Example 1); NMR (CDCl₃): 1.15 and 1.25 (t, 3H), 1.5 (s, 9H), 1.5 and 1.65 (m, 2H), 1.95 (m, 2H), 2.0-2.3 (m, 6H), 2.9 and 3.0 (m, 2H), 3.0 (s, 3H), 3.35 (ABq, 2H), 3.5 and 3.9 (m, 1H), 3.8 and 3.9 (s, 2H), 4.4 (m, 1H), 6.55 (br s, 1H), 6.8-7.0 (m, 4H), 7.1-7.3 (m, 4H), 7.5 (m, 2H), 7.9 (d, 2H); MS: 652 (MH+).

### EXAMPLE 14

This Example illustrates the preparation of N-[1-(3-phenyl-3-[4-methanethiophenyl]propyl)-piperidin-4-yl]-N-ethyl-4-methanesulfonylphenylacetamide (Compound No. 41 of Table I).

This was prepared from 3-phenyl-3-(4-methanethiophenyl)propionaldehyde (Method M) and N-(4-piperidinyl)-N-ethyl-4-methanesulfonylphenylacetamide using a method similar to that used to prepare N-[1-(3-phenyl-3-[4-phenylphenyl]propyl)-piperidin-4-yl]-N-ethyl-4 methanesulfonylphenylacetamide (Example 1); NMR (CDCl₃): 1.15 and 1.25 (t, 3H), 1.5 and 1.65 (m, 2H), 1.95 (m, 2H), 2.1-2.4 (m, 6H), 2.4 (s, 3H), 2.95 and 3.1 (m, 2H), 3.0 (s, 3H), 3.35 (ABq, 2H), 3.5 and 3.9 (m, 1H), 3.8 and 3.9 (s, 2H), 4.4 (m, 1H), 7.2 (m, 9H), 7.4 (m, 2H), 7.85 (d, 2H); MS: 565 (MH+).

### EXAMPLE 15

This Example illustrates the preparation of N-[1-(3-phenyl-3-[4-methanesulfonylphenyl]propyl)-piperidin-4-yl]-N-ethyl-4-methanesulfonylphenylacetamide (Compound No. 40 of Table I).

To a stirred solution of N-[1-(3-phenyl-3-[4-methanethiophenyl]propyl)-piperidin-4-yl]-N-ethyl-4-methanesulfonylphenylacetamide (example 14, 0.33 g, 0.58 mmol) in DCM (50 mL) was added 3-chloroperbenzoic acid (0.5 g, 2.92 mmol) and the resulting mixture was stirred at room temperature for 2 h. The mixture was washed with water, dried (MgSO₄), pre-absorbed onto a Bond Elut, and eluted with a gradient of DCM to 1% ammonia/10% methanol in DCM giving a white foam (0.205 g); MS: 613. This was dissolved in DCM (5 mL) and the solution cooled to 0° C. To this solution was added a mixture prepared as follows: formic acid (0.03 mL, 0.75 mmol) was dropwise to acetic anhydride (0.06 mL, 0.625 mmol) and the resulting mixture heated to 55° C. for 2 h then cooled. The resulting mixture was stirred at room temperature for 48 h. Water was added and the mixture basified with potassium carbonate to pH of approximately 10. The organic phase was dried (MgSO₄), pre-absorbed onto a Bond Elut and eluted with a gradient of DCM to 1% ammonia/10% methanol in DCM giving the title compound (0.12 g); NMR (CDCl₃): 1.15 and 1.25 (t, 3H), 1.5 and 1.65 (m, 2H), 1.6-2.0 (m, 4H), 2.25 (m, 4H), 2.9 and 3.0 (m, 2H), 3.0 (s, 3H), 3.05 (s, 3H), 3.35 (ABq, 2H), 3.5 and 3.8 (m, 1H), 3.8 and 4.1 (s, 2H), 4.4 (m, 1H), 7.2 (m, 5H), 7.45 (m, 4H), 7.8 (d, 2H), 7.9 (d, 2H); MS: 597 (MH+).

Below is presented certain NMR data for some compounds of the invention.

(S)-N-[1-{3-phenyl-3-(4-methanesulfonylphenyl)propyl}-4-piperidinyl]-N-ethyl-4 methanesulfonylphenylacetamide (Compound No. 50 of Table I).

NMR (CDCl₃): 1.15 and 1.25 (t, 3H), 1.5 and 1.65 (m, 2H), 1.6-2.0 (m, 4H), 2.25 (m, 4H), 2.9 and 3.0 (m, 2H), 3.0 (s, 3H), 3.05 (s, 3H), 3.35 (ABq, 2H), 3.5 and 3.8 (m, 1H), 3.8 and 4.1 (s, 2H), 4.4 (m, 1H), 7.2 (m, 5H), 7.45 (m, 4H), 7.8 (d, 2H), 7.9 (d, 2H)

(S)-N-[1-{3-(4-fluorophenyl)-3-(4-methanesulfonylphenyl)propyl}-4-piperidinyl]-N-ethyl-4-methanesulfonylphenylacetamide hydrochloride (Compound No. 52 of Table I).

NMR (d6-DMSO, 120° C.): 1.13 (t, 3H), 1.65 (m, 1H), 1.75 (m, 2H), 2.40 (m, 1H), 2.61 (m, 2H), 2.9-3.1 (m, 4H), 3.14 (s, 6H), 3.3-3.4 (m, 4H), 3.83 (s, 2H), 4.15 (m, 1H), 4.25 (dd, 1H), 7.10 (dd, 2H), 7.32 (dd, 2H), 7.40 (d, 2H), 7.50 (d, 2H), 7.85 (m, 4H), 11.1 (br s, 1H).

(R)-N-[1-{3-(3-chlorophenyl)-3-(4-methanesulfonylphenyl)propyl}-4-piperidinyl]-N-ethyl-4-methanesulfonylphenylacetamide hydrochloride (Compound No. 53 of Table I).

NMR (d6-DMSO, 120° C.): 1.13 (t, 3H), 1.65 (m, 1H), 1.75 (m, 2H), 2.40 (m, 1H), 2.61 (m, 2H), 2.9-3.1 (m, 4H), 3.14 (s, 3H), 3.3-3.4 (m, 7H), 3.83 (s, 2H), 4.20 (m, 1H), 4.30 (dd, 1H), 7.25 (m, 1H), 7.32 (m, 2H), 7.40 (s, 1H), 7.50 (d, 2H), 7.60 (d, 2H), 7.85 (m, 4H), 11.3 (br s, 1H).

(R)-N-[1-{3-(3,4-fluorophenyl)-3-(4-methanesulfonylphenyl)propyl}-4-piperidinyl]-N-ethyl-4-methanesulfonylphenylacetamide hydrochloride (Compound No. 54 of Table I).

NMR (d6-DMSO, 120° C.): 1.13 (t, 3H), 1.65 (m, 1H), 1.75 (m, 2H), 2.40 (m, 1H), 2.61 (m, 2H), 2.9-3.1 (m, 4H), 3.14 (s, 6H), 3.3-3.4 (m, 4H), 3.90 (s, 2H), 4.25 (m, 1H.), 4.35 (dd, 1H), 7.25 (m, 1H), 7.32 (dd, 1H), 7.45 (dd, 1H), 7.52 (d, 2H), 7.63 (d, 2H), 7.85 (m, 4H), 11.3 (br s, 1H).

(R)-N-[1-{3-(3,5-difluorophenyl)-3-(4-methanesulfonylphenyl)propyl}-4-piperidinyl]-N-ethyl-4-methanesulfonylphenylacetamide hydrochloride (Compound No. 56 of Table I).

NMR (d6-DMSO, 120° C.): 1.13 (t, 3H), 1.35 (m, 2H), 1.75 (m, 2H), 2.40 (m, 2H), 2.61 (m, 2H), 2.9-3.1 (m, 4H), 3.14 (s, 6H), 3.35 (q, 2H), 3.45 (m, 2H), 3.87 (s, 2H), 4.15 (m, 1H), 4.35 (dd, 1H), 6.95 (t, 1H), 7.10 (d, 2H), 7.50 (d, 2H), 7.63 (d, 2H), 7.85 (m, 4H), 11.2 (br s, 1H).

Method A

N-(4-Piperidinyl)-N-ethyl-4-methanesulfonylphenylacetamide

Step 1: Preparation of 1-phenylmethyl-4-ethylaminopiperidine Dihydrochloride

To a solution of 1-phenylmethyl-4-piperidone (25.0 g, 132 mmol) in THF (250 mL) was added ethylamine hydrochloride (12.0 g, 147 mol) and methanol (50 mL) and the resulting mixture stirred at room temperature for 10 min. Sodium triacetoxyborohydride (40 g, 189 mmol) was added portionwise and the resulting mixture stirred at room temperature for 1 h. 2M Sodium hydroxide solution (250 mL) was added and the resulting mixture extracted with diethyl ether. The organic extracts were dried (K₂CO₃) and evaporated to give 1-phenylmethyl-4-ethylaminopiperidine as an oil. This was dissolved in ethanol (500 mL) and concentrated hydrochloric acid (20 mL) was added. The resulting crystals were collected, washed with diethyl ether and dried giving the sub-titled compound as a solid (38 g); NMR: (CDCl₃): 1.10 (t, 3H), 1.40 (m, 2H), 1.83 (m, 2H), 2.02 (m, 2H), 2.65 (q, 2H), 2.85 (m, 2H), 3.50 (s, 2H), 3.75 (m, 1H), 7.2-7.4 (m, 5H); MS: 219 (MH+).

Step 2: Preparation of N-(1-Phenylmethyl-4-piperidinyl)-N-ethyl-4-methanesulfonylphenylacetamide

To a solution of 1-phenylmethyl ethylaminopiperidine dihydrochloride (32.0 g, 110 mmol) in DCM (500 mL) was added N₃N-diisopropylethylamine (60 mL) with stirring to ensure complete dissolution. 4-Methanesulfonylphenylacetic acid (25.0 g, 117 mmol), 4-Dimethylaminopyridine (2.0 g) and dicyclohexylcarbodiimide (25.0 g, 121 mmol) were added and the resulting mixture was stirred at room temperature for 20 h. The precipitate was removed by filtration and the resulting solution was washed successively with 2N aqueous HCl, water and 1N aqueous NaOH, dried (MgSO₄) and evaporated. The residue was purified by silica gel chromatography (eluent: 10% MeOH/ethyl acetate) to afford the sub-titled compound (35 g, 76%); NMR: 1.00 and 1.14 (t, 3H), 1.45 and 1.70 (m, 2H), 1.95 (br m, 2H), 2.80 (br m, 2H), 3.18 (s, 3H), 3.20 and 3.33 (q, 2H), 3.45 (s, 2H), 3.80 and 3.87 (s, 2H), 3.70 and 4.10 (m, 1H), 7.2-7.3 (m, 5H), 7.48 (m, 2H), 7.82 (m, 2H); MS: 415 (MH+).

Step 3: Preparation of the Title Compound

To a solution of N-(1-phenylmethyl-4-piperidinyl)-N-ethyl-4-methanesulfonylphenyl-acetamide (34 g, 82 mmol) in ethanol (600 mL) was added ammonium formate (40 g). The mixture was purged with argon and 30% Pd on carbon (4.2 g) was added. The resulting mixture was stirred at reflux for 4 h, then allowed to cool and filtered through diatomaceous earth. The filtrate was evaporated to give a thick oil which solidified on standing to yield the title compound (24.9 g, 94%); NMR: 1.02 and 1.15 (t, 3H), 1.4-1.6 (br m, 4H), 2.45 (m, 2H), 2.93 (br m, 2H), 3.18 (s, 3H), 3.20 and 3.32 (q, 2H), 3.72 and 4.18 (m, 1H), 3.80 and 3.87 (s, 2H), 7.50 (m, 2H), 7.85 (m, 2H); MS: 325 (MH+).

Method B

3-Phenyl-3-(4-phenylphenyl)propionaldehyde

Step 1: Preparation of Ethyl 3-phenyl-3-(4-phenylphenyl)acrylate

To a solution of triethylphosphonoacetate (6.7 g, 26 mmol) in THF (100 mL) at 0° C. was added lithium bis(trimethylsilyl)amide (26 mL, 1M, 26 mmol). The resulting mixture was stirred at 0° C. for 20 min. 4-Benzoylbiphenyl (6.5 g, 26 mmol) was added and the resulting mixture was stirred at room temperature for 48 h. The mixture was evaporated and the residue dissolved in ethyl acetate (200 mL). The solution was washed with 2M hydrochloric acid (2×100 mL), dried and evaporated giving the sub-titled compound (11 g).

Step 2: Preparation of Ethyl 3-phenyl-3-(4-phenylphenyl)propionoate

Ethyl 3-phenyl-3-(4-phenylphenyl)acrylate (11 g) was dissolved in ethanol (200 mL) and the solution purged with argon. 20% Palladium hydroxide (2 g) was added and the resulting mixture was stirred at room temperature under an atmosphere of hydrogen (balloon) for 72 h. The mixture was purged with argon, filtered and the filtrate evaporated. The crude product was purified by silica gel chromatography (eluent: isohexane then 25% ethyl acetate in isohexane) to give the sub-titled compound (2.8 g).

Step 3: Preparation of 3-phenyl-3-(4-phenylphenyl)propan-1-ol

To a solution of ethyl 3-phenyl-3-(4-phenylphenyl)propionoate (2.8 g, 8.48 mmol) in THF (30 mL) was added lithium aluminium hydride (8.48 mL, 1M, 8.48 mmol) dropwise over 30 min. The resulting mixture was stirred at 0° C. for 1 h. 2M Aqueous sodium hydroxide (8 mL) was added dropwise. The mixture was filtered through Celite®, washing with ethyl acetate (3×25 mL). The filtrate and washings were combined and evaporated. The residue was dissolved in ethyl acetate (50 mL) and the resulting solution washed with water (50 mL) and 2M hydrochloric acid (2×50 mL), dried and evaporated. The residue was purified by silica gel chromatography (eluent: isohexane then 40% ethyl acetate in isohexane) to give the sub-titled compound (1.3 g); NMR: 2.2 (q, 2H) 3.3 (q, 2H) 4.1 (t, 1H) 4.25 (t, 1H) 7.1-7.6 (m, 14H).

Step 4: Preparation of the Title Compound

To a solution of 3-phenyl-3-(4-phenylphenyl)propan-1-ol (1.3 g, 3.3 mmol) in DCM (50 mL) was added Dess-Martin periodinane (1.8 g, 4.4 mmol) and the resulting mixture was stirred at room temperature for 1.5 h. The mixture was washed with 2M aqueous sodium hydroxide (2×20 mL), dried and evaporated to give the title compound (1.3 g); NMR: 3.2 (d, 2H) 4.6 (t, 1H) 7.1-7.7 (m, 14H) 9.7 (s, 1H).

Method C

3-Phenyl-3-(4-Boc-aminophenyl)propionaldehyde

This was prepared from 4-nitrobenzophenone using a similar sequence of reactions to that used to prepare 3-phenyl-3-(4-phenylphenyl)propanionaldehyde from 4-benzoylbiphenyl (Method B), except that an additional step was included between Steps 2 and 3, namely treatment of ethyl 3-phenyl-3-(4-aminophenyl)propionate with di-tert-butyldicarbonate to form ethyl 3-phenyl-3-(4-Boc-aminophenyl)propionate.

Method D

E-(4R, 5S)-1-(3-[4-Methanesulfonylphenyl]acryloyl)-3,4-dimethyl-5-phenyl-imidazolidin-2-one

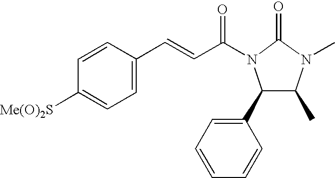

To a stirred solution of 3-(4-methanesulfonylphenyl)acrylic acid (7.14 g, 31.5 mmol) in DCM (10 mL) was added thionyl chloride (3 mL, 34.7 mmol) dropwise and the resulting mixture was stirred at room temperature for 18 h. To this solution was added DIPEA (5.04 mL, 28.9 mmol) dropwise at room temperature. The resulting solution was added to a stirred solution of (4R, 5S)-3,4-dimethyl-5-phenyl-imidazolidin-2-one (5.0 g, 26.3 mmol) in DCM (20 mL) and DIPEA (4.58 mL, 26.9 mmol) and the resulting mixture stirred at room temperature for 4 h. The mixture was washed with water and brine, pre-absorbed onto a Bond Elut and eluted with a gradient of isohexane to ethyl acetate giving the title compound as a solid (7.61 g, 73%); NMR (CDCl₃): 0.84 (d, 3H), 2.89 (s, 3H), 3.04 (s, 3H), 3.98 (m, 1H), 5.42 (d, 1H), 7.20 (m, 2H), 7.32 (m, 3H), 7.69 (d, 1H), 7.74 (d, 2H), 7.93 (d, 2H), 8.31 (d, 1H); MS: 399 (MH+).

Method E

4-Methanethiobenzophenone

Step 1: Preparation of 1-(4-methanethiophenyl)phenylmethanol

To a solution of 4-methanethiobenzaldehyde (21 g, 138 mmol) in THF (200 mL) at 0° C. was added phenyl lithium (84 mL, 152 mmol) dropwise. The resulting mixture was stirred for 18 h with warming to room temperature. The mixture was washed with saturated aqueous ammonium chloride and brine, dried (MgSO₄) and evaporated giving the sub-tided compound as a solid (30.16 g, 95%); NMR (CDCl₃): 2.28 (dt, 2H), 2.43 (s, 3H), 3.58 (t, 2H), 4.10 (t, 1H), 7.23 (m, 5H).

Step 2: Preparation of Title Compound

To a solution of 1-(4-methanethiophenyl)phenylmethanol (30 g, 130 mmol) in DCM (400 mL) was added Dess-Martin periodinane (55 g, 143 mmol) portionwise at room temperature. The resulting mixture was stirred at room temperature for 1 h, washed with 2M aqueous sodium hydroxide, dried (MgSO₄), pre-absorbed onto a silica column and eluted with a gradient elution (isohexane to DCM) to give the title compound as a solid (18.57 g, 63%); NMR (CDCl₃): 2.53 (s, 3H), 7.29 (m, 2H), 7.48 (m, 2H), 7.58 (m, 1H), 7.76 (m, 4H); MS: 229 (MH+).

Method F

3-Phenyl-3-(4-methoxycarbonylphenyl)propionaldehyde

Step 1: Preparation of diphenylmethane-4-carboxylic Acid Methyl Ester

To a suspension of diphenylmethane-4-carboxylic acid (10 g, 47 mmol) in methanol (50 mL) was added thionyl chloride (0.34 mL, 4.7 mmol) dropwise. The resulting mixture was heated to reflux for 3 h then allowed to cool. The mixture was evaporated and eluted through a plug of silica gel to give the sub-titled compound (9.7 g, 91%); NMR (CDCl₃): 3.9 (s, 3H), 4.0 (s, 2H), 7.2 (m, 7H) and 8.0 (d, 2H).

Step 2: Preparation of 3-phenyl-3-(4-methoxycarbonylphenyl)but-1-ene

To a solution of diphenylmethane-4-carboxylic acid methyl ester (9.7 g, 43 mmol) in THF (100 mL) under argon at −78° C. was added lithium diisopropylamide (23.5 mL, 2M, 47 mmol) dropwise and the resulting mixture was stirred at −78° C. for 1 h. Allyl bromide (1.85 mL, 21 mmol) was added and the resulting mixture was allowed to warm to room temperature over 18 h. The reaction mixture was washed with water and brine, dried (MgSO₄) and evaporated. The residue was purified by silica gel chromatography (eluent: DCM) to give the subtitled compound as an oil (4.3 g); NMR (CDCl₃): 2.87 (dd, 2H), 3.91 (s, 3H), 4.12 (t, 1H), 5.03 (m, 2H), 5.76 (m, 1H), 7.24 (m, 7H), 8.01 (d, 2H).

Step 3: Preparation of the Title Compound

A solution of 3-phenyl-3-(4-methoxycarbonylphenyl)but-1-ene (3.26 g, 12.2 mmol) in methanol (100 mL) was purged with oxygen gas at −78° C. for 10 min. Ozone was bubbled through for 1 h until a blue colour persisted. Dimethyl sulfide (1.8 mL, 25 mmol) was added and the resulting mixture was stirred at room temperature for 1 h then evaporated to give the title compound which was used in the next reaction without further purification.

Method G

3-Phenyl-3-(pyridin-2-yl)propionaldehyde

This was prepared from 2-benzylpyridine using a similar method to that used to prepare 3-phenyl-3-(4-methoxycarbonylphenyl)propionaldehyde from diphenylmethane-4-carboxylic acid methyl ester (Method F).

Method H

N-[1-(3-phenyl-3-[4-carboxyphenyl]propyl)-piperidin-4-yl]-N-ethyl-4-methanesulfonylphenylacetamide Hydrochloride

To a solution of N-[1-(3-phenyl-3-[4-methoxycarbonylphenyl]propyl)-piperidinyl]-N-ethyl-4-methanesulfonylphenylacetamide (Example 2; 2.36 g, 4.1 mmol) in methanol (40 mL) was added sodium hydroxide (1.64 g, 41 mmol) and the resulting mixture was stirred at room temperature for 3 days. The mixture was evaporated, the residue was taken up in water and acidified to pH 1. The solid was collected and triturated with methanol to give the title compound (0.73 g); NMR: 1.0 and 1.1 (t, 3H), 1.4 (m, 2H), 1.6 (m, 2H), 1.8 (br t, 2H), 2.2 (m, 4H), 2.8 (br t, 2H), 3.2 (s, 3H), 3.3 (m, 1H), 3.6 (m, 1H), 3.8 (d, 2H), 4.0 (m, 2H), 7.1 (m, 1H), 7.3 (m, 4H), 7.5 (m, 4H) and 7.8 (d, 4H); MS: 563.

Method I

3-(4-Chlorophenyl)-3-(3-fluorophenyl)propionaldehyde

This was prepared from 4-chloro-3′-fluorobenzophenone (Method N) using a similar sequence of reactions to that used to prepare 3-phenyl-3-(4-phenylphenyl)propanionaldehyde from 4-benzoylbiphenyl (Method B), except that Step 2 was omitted.

Method J

4-Chloro-3′-fluorobenzophenone

Step 1: Preparation of 4-chlorophenyl-3-fluorophenylmethanol

To a solution of 3-fluorobenzaldehyde (5 g, 40 mmol) in THF (20 mL) was added 4-chlorophenylmagnesium bromide (44 mL, 1M in diethyl ether, 44 mmol) dropwise and the resulting mixture was stirred at room temperature for 1 h. 2M Hydrochloric acid (10 mL) was added portionwise, the layers separated and the organic phase evaporated. The residue was purified by Bond Elut chromatography (eluent: isohexane then DCM) to give the sub-titled compound (5.1 g, 48%); NMR (CDCl₃): 2.2 (d, 1H), 5.8 (d, 1H), 7.0 (m, 1H), 7.1 (m, 2H) and 7.3 (m, 5H).

Step 2: Preparation of the Title Compound

To a solution of 4-chlorophenyl-3-fluorophenylmethanol (1.55 g, 6.55 mmol) in DCM (40 mL) was added Dess-Martin periodinane (3.06 g, 7.20 mmol) and the resulting mixture was stirred at room temperature for 30 min. The reaction mixture was washed with 2M aqueous sodium hydroxide, dried and evaporated. The residue was purified by Bond Elut chromatography (eluent: isohexane then DCM) to give the title compound as a white solid (1.00 g, 64%); NMR (CDCl₃): 7.3 (m, 1H), 7.5 (m, 5H) and 7.8 (d, 2H).

Method K

(2R)-1-(4-[4-Chlorophenyl]-4-[3-fluorophenyl]but-2-yl)-4-ethylaminopiperidine 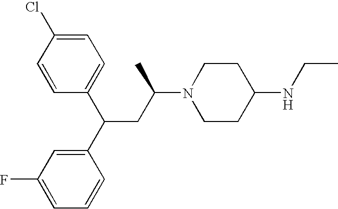
Step 1: Preparation of 4-chloro-3′-fluorodiphenylmethane

To a mixture of 4-chlorophenyl-3-fluorophenylmethanol (Step1 of Method J; 10 g, 38 mmol) and glacial acetic acid (150 mL) was added iodine (10 g, 40 mmol) and hypophosphoric acid (30 mL, 50% aqueous, 285 mmol). The resulting mixture was stirred at 60° C. for 16 h, allowed to cool and diluted with water (300 mL). The mixture was extracted twice with isohexane and the combined extracts washed with saturated aqueous sodium bicarbonate solution, dried (MgSO₄) and evaporated. The residue was purified by silica gel chromatography (eluent: isohexane) to give the sub-titled compound (7.5 g); NMR (CDCl₃): 3.9 (s, 2H), 6.9 (m, 3H), 7.1 (d, 2H) and 7.2 (m, 3H); MS: 220 (MH+).
Step 2: Preparation of (1R)-N-tosyl-3-(4-chlorophenyl)-3-(3-fluorophenyl)-1-methylpropylamine

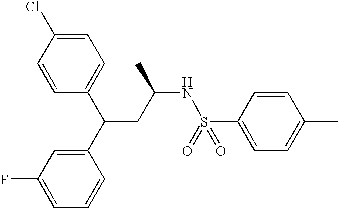

To a solution of 4-chloro-3′-fluorodiphenylmethane (7.0 g, 32 mmol) in TH (100 mL) at −10° C. was added lithium diisopropylamide (20 mL, 2M in hexane/TBF, 40 mmol) and the resulting mixture was stirred at −10° C. for 10 min. during which time a deep red colour was observed. (R)-2-Methyl-1-tosylaziridine (prepared from D-alaninol in two steps using literature procedures; 6.7 g, 32 mmol) was added and the resulting mixture was stirred with warming to room temperature for 1 h. The reaction mixture was partitioned between saturated aqueous ammonium chloride and diethyl ether, the combined organic phases were dried (MgSO₄) and evaporated to give the sub-titled compound as a solid (12 g); NMR (CDCl₃): 1.0 (dd, 3H), 2.0 (m, 1H), 2.1 (m, 1H), 2.4 (s, 3H), 3.1 (m, 1H), 4.0 (m, 1H), 4.6 (m, 1H), 6.8 (m, 1H), 6.9 (m, 1H), 7.2 (m, 8H) and 7.6 (m, 2H).
*Tetrahedron 44, 3919 (1988), Chem. Pharm. Bull. 25, 29 (1977).

Step 3: Preparation of (1R)-3-(4-chlorophenyl)-3-(3-fluorophenyl)-1-methylpropylamine

A mixture of (1R)-N-tosyl-3-(4-chlorophenyl)-3-(3-fluorophenyl)-1-methylpropylamine (10 g, 23 mmol) and 30% hydrobromic acid in glacial acetic acid (10 mL) was heated to 80° C. for 18 h, allowed to cool and evaporated. The residue was partitioned between diethyl ether and 2M aqueous sodium hydroxide. The organic phase was evaporated and the residue purified by silica gel chromatography (eluent: 2:1 ethyl acetate/methanol) to give the sub-titled compound (2.2 g); NMR (CDCl₃): 1.4 (d, 3H), 2.2 (m, 2H), 3.2 (br s, 2H), 4.3 (m, 1H) and 7.0 (br m, 8H): MS: 278 (MH+).
Step 4: Preparation of (2R)-1-(4-[4-chlorophenyl]-4-[3-fluorophenyl]but-2-yl)-4-piperidone

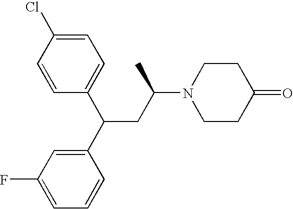

To a solution of (1R)-3-(4-chlorophenyl)-3-(3-fluorophenyl)-1-methylpropylamine (2.0 g 7.2 mmol) in ethanol (50 mL) was added a solution of potassium carbonate (1.5 g) in water (5 mL) and the resulting mixture heated to reflux with stirring. A solution of 1-methyl-1-ethyl-4-oxopiperidinium iodide (2.5 g, 9.3 mmol) in water (15 mL) was added dropwise. The resulting mixture was stirred at reflux for 10 min. then allowed to cool. The mixture was concentrated to about half the volume then extracted with DCM. The combined extracts were evaporated and the residue purified by silica gel chromatography (eluent: ethyl acetate) to give the sub-titled compound (700 mg); NMR: 0.9 (m, 3H), 2.0 (m, 1H), 2.2 (m, 1H), 2.3 (m, 4H), 2.4 (m, 5H), 2.7 (m, 2H), 7.0 (m, 1H), 7.2 (m, 2H) and 7.3 (m, 5H).

Step 5: Preparation of the Title Compound

To a solution of (2R)-1-(4-[4-chlorophenyl]-4-[3-fluorophenyl]but-2-yl)-4-piperidone (400 mg, 1.1 mmol) in ethanol (20 mL) was added ethylamine hydrochloride (200 mg, 2.5 mmol) and the resulting mixture was stirred at room temperature for 10 min. until dissolved. Sodium triacetoxyborohydride (400 mg, 1.88 mmol) was added and the resulting mixture was stirred at room temperature for 18 h. The reaction mixture was partitioned between 2M aqueous sodium hydroxide and diethyl ether. The organic phase was dried and evaporated and the residue purified by silica gel chromatography (eluent: 1% ammonia/10% methanol in DCM) to give the title compound (220 mg); MS: 389 (MH+).

Method L

3-(3-Fluorophenyl)-3-(4-Boc-aminophenyl)propionaldehyde

This was prepared from 4-nitro-3′-fluorobenzophenone using a similar sequence of reactions to that used to prepare 3-phenyl-3-(4-Boc-aminophenyl)propionaldehyde from 4-nitrobenzophenone (Method C).

Method M

3-Phenyl-3-(4-methanethiophenyl)propionaldehyde

This was prepared from 4-methanethiobenzophenone (Method E) using a similar sequence of reactions to that used to prepare 3-phenyl-3-(4-phenylphenyl)propanionaldehyde from 4-benzoylbiphenyl (Method B).

Method N

(S)-3-Phenyl-3-(4-methanesulfonylphenyl)propionaldehyde 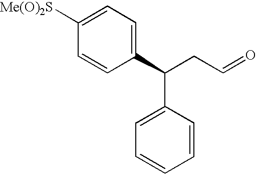
Step 1: Preparation of (4R, 5S)-1-[(S)-3-(4-methanesulfonyl-phenyl)-3-phenyl-propionyl]-3,4-dimethyl-5-phenyl-imidazolidin-2-one

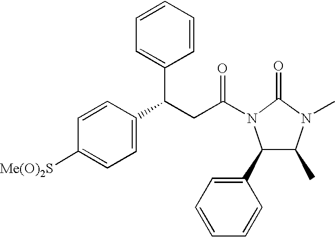

To a mixture of copper (I) iodide (960 mg, 5.0 mmol) and THF (20 mL) was added N,N,N′,N′-tetramethylethylenediamine (0.83 mL, 5.5 mmol) and the resulting mixture was stirred at room temperature for 10 min. then cooled to −78° C. Phenylmagnesium bromide (5.0 mL, 1M in THF, 5.0 mmol) was added and the resulting mixture stirred at −78° C. for 15 min. A solution of di-n-butylboron triflate (3.0 mL, 1M in diethyl ether, 3.0 mmol) and (E)-(4R, 5S)-1-(3-[4-methanesulfonylphenyl]acryloyl)-3,4-dimethyl-5-phenyl-imidazolidin-2-one (Method D, 1.0 g, 2.51 mmol) in THF (15 mL) was added and the resulting mixture was stirred whilst allowing to warm to room temperature for 18 h. The reaction mixture was washed with saturated aqueous ammonium chloride, water and brine, dried (MgSO₄) and evaporated. The residue was purified by eluting through a 20 g Bond Elut with gradient of isohexane to ethyl acetate giving the sub-titled compound (1.49 g, 100%); NMR (CDCl₃): 0.78 (d, 3H), 2.82 (s, 3H), 3.00 (s, 3H), 3.78 (dd, 1H), 3.80 (m, 1H), 3.98 (dd, 1H), 4.72 (m, 1H), 5.19 (d, 1H), 6.99 (m, 2H), 7.22 (m, 8H), 7.48 (d, 2H), 7.79 (d, 2H); MS: 477 (MH⁺).

Step 2: Preparation of (S)-3-phenyl-3-(4-methanesulfonylphenyl)propan-1-ol

To a solution of (4R, 5S)-1-[(S)-3-(4-methanesulfonyl-phenyl)-3-phenyl-propionyl]-3,4-dimethyl-5-phenyl-imidazolidin-2-one (846 mg, 1.78 mmol) in THF (20 mL) at 0° C. was added lithium aluminium hydride (3.6 mL, 1M in TBIF, 3.6 mmol) and the resulting mixture was stirred for 15 min. The reaction was quenched by the addition of 2M aqueous sodium hydroxide. The phases were separated and the organic phase pre-absorbed onto a Bond Elut and eluted with a gradient of isohexane to ethyl acetate giving the sub-titled compound as a white solid (285 mg, 55%); NMR (CDCl₃): 1.63 (br s, 1H), 2.33 (m, 2H), 3.00 (s, 3H), 3.59 (t, 2H), 4.28 (t, 1H), 7.23 (m, 5H), 7.43 (d, 2H), 7.82 (d, 2H).

Step 3: Preparation of the Title Compound

To a solution of (S)-3-phenyl-3-(4-methanesulfonylphenyl)propan-1-ol (244 mg, 0.84 mmol) in DCM (5 mL) was added Dess-Martin periodinane (392 mg, 0.92 mmol) and the resulting mixture was stirred at room temperature for 1.5 h. The mixture was washed with 2M aqueous sodium hydroxide (2×10 mL), dried and evaporated to give the title compound.

Method O

(R)-3-(3-fluorophenyl)-3-(4-methanesulfonylphenyl)propionaldehdye

This was prepared from (4R, 5S)-1-(3-[4-methanesulfonylphenyl]acryloyl)-3,4-dimethyl-5-phenyl-imidazolidin-2-one and 3-fluorophenylmagnesium bromide using a method similar to that used to prepare (S)-3-phenyl-3-(4-methanesulfonyl-phenyl)propionaldehyde from phenylmagnesium bromide (Method N); NMR (CDCl₃): 3.01 (s, 3H), 3.24 (d, 2H), 4.73 (t, 1H), 6.91 (m, 2H), 6.99 (m, 1H), 7.28 (m, 2), 7.42 (d, 2H), 7.87 (d, 2H), 9.76 (s, 1H).

Method P

(S)-3-(4-fluorophenyl)-3-(4-methanesulfonylphenyl)propionaldehdye

This was prepared from (4R, 5S)-1-(3-[4-methanesulfonylphenyl]acryloyl)-3,4-dimethyl-5-phenyl-imidazolidin-2-one and 4-fluorophenylmagnesium bromide using a method similar to that used to prepare (S)-3-phenyl-3-(4-methanesulfonyl-phenyl)propionaldehyde from phenylmagnesium bromide (Method N).

Method Q

(R)-3-(3-chlorophenyl)-3-(4-methanesulfonylihenyl)propionaldehdye

This was prepared from (4R, 5S)-1-(3-[4-methanesulfonylphenyl]acryloyl)-3,4-dimethyl-5-phenyl-imidazolidin-2-one and 3-chlorophenylmagnesium bromide using a method similar to that used to prepare (S)-3-phenyl-3-(4-methanesulfonyl-phenyl)propionaldehyde from phenylmagnesium bromide (Method N).

Method R

(R)-3-(3,4-difluorophenyl)-3-(4-methanesulfonylphenyl)propionaldehdye

This was prepared from (4R, 5S)-1-(3-[4-methanesulfonylphenyl]acryloyl)-3,4-dimethyl-5-phenyl-imidazolidin-2-one and 3,4-difluorophenylmagnesium bromide using a method similar to that used to prepare (S)-3-phenyl-3-(4-methanesulfonyl-phenyl)propionaldehyde from phenylmagnesium bromide (Method N).

Method S

(S)-3-(4-methoxyphenyl)-3-(4-methanesulfonylphenyl)propionaldehdye

This was prepared from (4R, 5S)-1-(3-[4-methanesulfonylphenyl]acryloyl)-3,4-dimethyl-5-phenyl-imidazolidin-2-one and 4-methoxyphenylmagnesium bromide using a method similar to that used to prepare (S)-3-phenyl-3-(4-methanesulfonyl-phenyl)propionaldehyde from phenylmagnesium bromide (Method N).

Method T

(R)-3-(3,5-difluorophenyl)-3-(4-methanesulfonylhenyl)propionaldehdye

This was prepared from (4R, 5S)-1-(3-[4-methanesulfonylphenyl]acryloyl)-3,4-dimethyl-5-phenyl-imidazolidin-2-one and 3,5-difluorophenylmagnesium bromide using a method similar to that used to prepare (S)-3-phenyl-3-(4-methanesulfonyl-phenyl)propionaldehyde from phenylmagnesium bromide (Method N).

### EXAMPLE 16

The ability of compounds to inhibit the binding of RANTES was assessed by an in vitro radioligand binding assay. Membranes were prepared from Chinese hamster ovary cells which expressed the recombinant human CCR5 receptor. These membranes were incubated with 0.1 nM iodinated RANTES, scintillation proximity beads and various concentrations of the compounds of the invention in 96-well plates. The amount of iodinated RANTES bound to the receptor was determined by scintillation counting. Competition curves were obtained for compounds and the concentration of compound which displaced 50% of bound iodinated RANTES was calculated (IC₅₀). Preferred compounds of formula (1) have an IC₅₀ of less than 50 μM.

### EXAMPLE 17

The ability of compounds to inhibit the binding of MIP-1 α was assessed by an in vitro radioligand binding assay. Membranes were prepared from Chinese hamster ovary cells which expressed the recombinant human CCR5 receptor. These membranes were incubated with 0.1 nM iodinated MIP-1α, scintillation proximity beads and various concentrations of the compounds of the invention in 96-well plates. The amount of iodinated MIP-1α bound to the receptor was determined by scintillation counting. Competition curves were obtained for compounds and the concentration of compound which displaced 50% of bound iodinated MIP-1α was calculated (IC₅₀). Preferred compounds of formula (1) have an IC₅₀ of less than 50 μM.

Results from this test for certain compounds of the invention are presented in Table II. In Table II the results are presented as Pic50 values. A Pic50 value is the negative log (to base 10) of the IC₅₀ result, so an IC50 of 1 μM. (that is 1×10⁻⁶ M) gives a Pic50 of 6. If a compound was tested more than once then the data below is an average of the probative tests results.

TABLE II

| Compound No. | Pic50 |
|---|---|
| 28 | 6.07 |
| 40 | 8.77 |
| 41 | 7.93 |
| 42 | 7.88 |
| 43 | 7.05 |
| 44 | 6.98 |
| 45 | 8.44 |
| 46 | 8.04 |
| 47 | 7.46 |
| 48 | 6.51 |
| 49 | 8.21 |
| 50 | 9.09 |
| 51 | 9.43 |
| 52 | 7.34 |
| 53 | 9 |
| 54 | 7.67 |
| 55 | 6.88 |
| 75 | 7.86 |

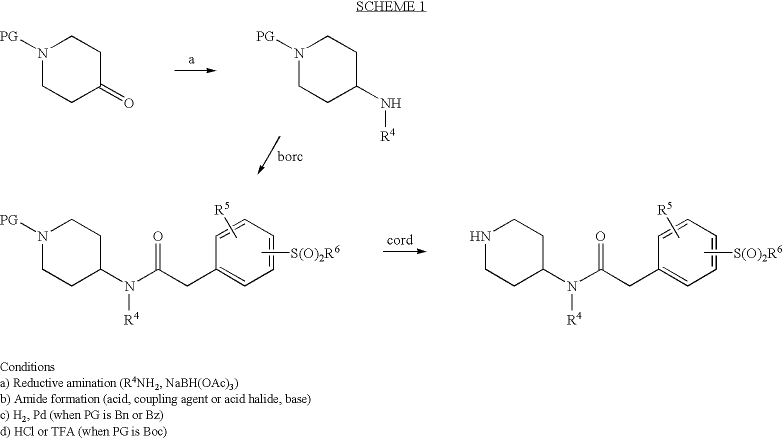

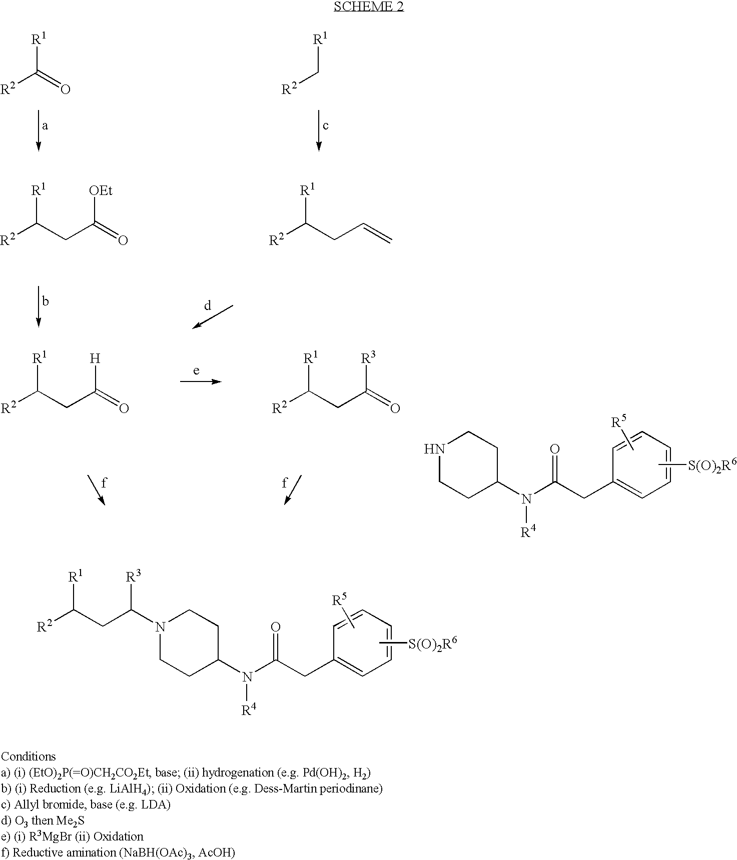

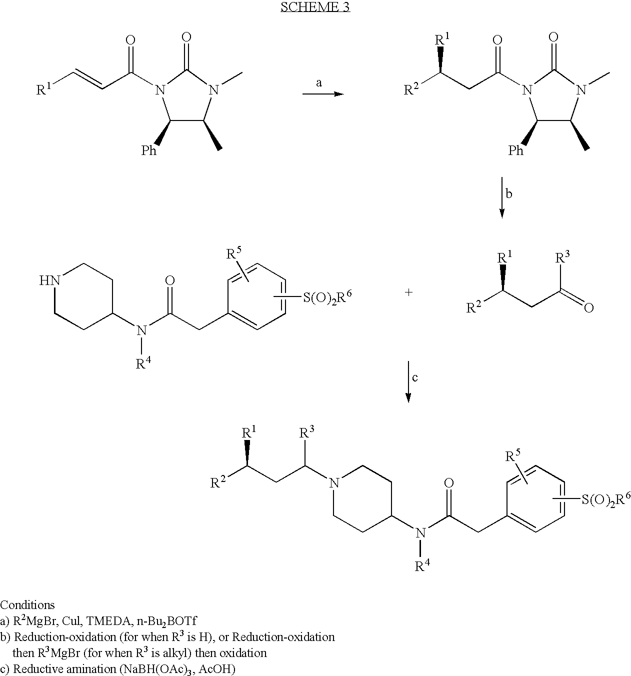

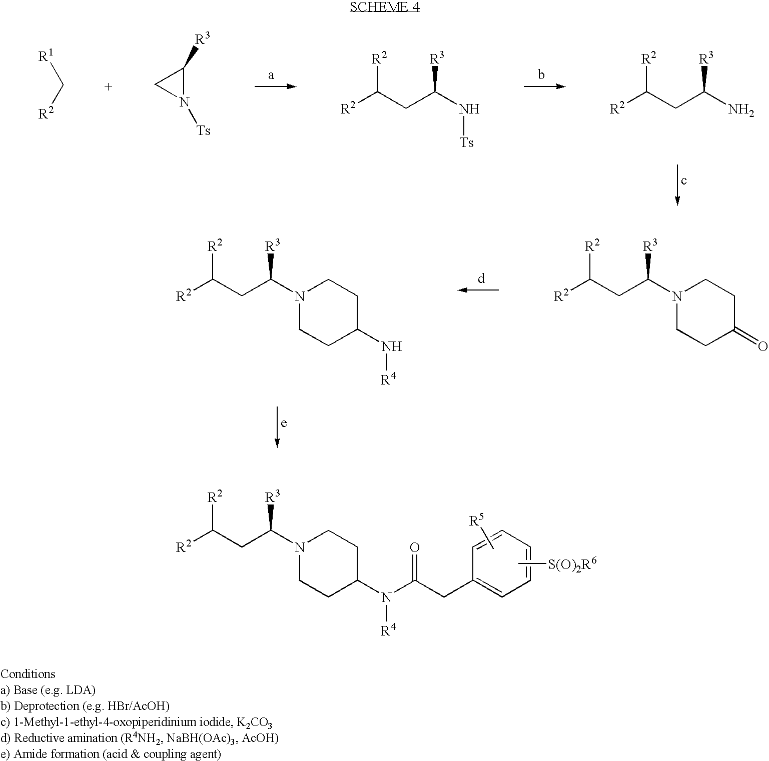

1. A compound of formula (I): 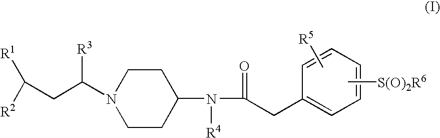
wherein:
R¹ is phenyl {para-substituted by: halo, hydroxy, nitro, S(O)ₖ(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, C(O)[N-linked heterocyclyl], CO₂H, CO₂(C₁₋₆ alkyl), NHC(O)(C₁₋₆ alkyl), NHC(O)O(C₁₋₆ alkyl), NHS(O)₂(C₁₋₆ alkyl), C(O)(C₁₋₆ alkyl), CF₃, OCF₃, phenyl, heteroaryl, (C₁₋₄ alkyl)phenyl, (C₁₋₄ alkyl)heteroaryl, NHC(O)phenyl, NHC(O)heteroaryl, NHC(O)(C₁₋₄ alkyl)phenyl, NHC(O)(C₁₋₄ alkyl)heteroaryl, NHS(O)₂phenyl, NHS(O)₂heteroaryl, NHS(O)₂(C₁₋₄ alkyl)phenyl, NHS(O)₂(C₁₋₄ alkyl)heteroaryl, NHC(O)NH(C₁₋₆ alkyl), NHC(O)NH(C₃₋₇ cycloalkyl), NHC(O)NHphenyl, NHC(O)NHheteroaryl, NHC(O)NH(C₁₋₄ alkyl)phenyl or NHC(O)NH(C₁₋₄ alkyl)heteroaryl; wherein the foregoing phenyl and heteroaryl groups are optionally substituted by halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃};
R² is phenyl or heteroaryl, either of which is optionally substituted by halo, C₁₋₄ alkyl,
C₁₋₄ alkoxy, S(O)ₙ(C₁₋₄ alkyl), nitro, cyano or CF₃;
R³ is hydrogen or C₁₋₄ alkyl;
R⁴ is ethyl, allyl or cyclopropyl;
R⁵ is hydrogen, halo, hydroxy, nitro, S(O)ₘ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
R⁶ is C₁₋₄ alkyl;
k, m and n are, independently, 0, 1 or 2;
or a pharmaceutically acceptable salt thereof or a solvate thereof;
provided that:
when R³ and R⁵ are both hydrogen, R⁴ is ethyl, R⁶ is para-(S(O)₂CH₃) and R² is unsubstituted phenyl then R¹ is not para-methoxy-phenyl, para-methyl-phenyl, para-trifluoromethyl-phenyl or 3,4-dichlorophenyl;
when R³ and R⁵ are both hydrogen, R⁴ is ethyl, R⁶ is para-(S(O)₂CH₃) and R² is unsubstituted phenyl, pyrid-2-yl or pyrid-4-yl then R¹ is not para-chloro-phenyl; and,
when R³ and R⁵ are both hydrogen, R⁶ is para-(S(O)₂CH₃) and R² is meta-chloro-phenyl, unsubstituted phenyl or thiophen-3-yl then R¹ is not para-fluoro-phenyl.

2. A compound as claimed in claim 1 wherein R¹ is phenyl {para-substituted by: halo, S(O)ₖ(C₁₋₆ alkyl), S(O)₂NH₂, S(O)₂NH(C₁₋₆ alkyl), S(O)₂N(C₁₋₆ alkyl)₂, cyano, NH₂, NH(C₁₋₆ alkyl), N(C₁₋₆ alkyl)₂, CO₂(C₁₋₆ alkyl), NHC(O)(C₁₋₆ alkyl), NHC(O)O(C₁₋₆ alkyl), NHS(O)₂(C₁₋₆ alkyl), NHC(O)phenyl, NHC(O)heteroaryl, NHC(O)(C₁₋₄ alkyl)phenyl, NHC(O)(C₁₋₄ alkyl)heteroaryl, NHS(O)₂phenyl, NHS(O)₂heteroaryl, NHS(O)₂(C₁₋₄ alkyl)phenyl, NHS(O)₂(C₁₋₄ alkyl)heteroaryl, NHC(O)NH(C₁₋₆ alkyl), NHC(O)NH(C₃₋₇ cycloalkyl), NHC(O)NHphenyl, NHC(O)NHheteroaryl, NHC(O)NH(C₁₋₄ alkyl)phenyl or NHC(O)NH(C₁₋₄ alkyl)heteroaryl; wherein the foregoing phenyl and heteroaryl groups are optionally substituted by halo, hydroxy, nitro, S(O)ₖ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃}; and k is 2.

3. A compound as claimed in claim 1 wherein R² is phenyl, mono-fluorophenyl, difluorophenyl, mono-chlorophenyl or mono-(C₁₋₄ alkoxy)phenyl.

4. A compound as claimed in claim 1 wherein R³ is hydrogen.

5. A compound as claimed in claim 1 wherein R⁴ is ethyl.

6. A compound as claimed in claim 1 wherein R⁵ is hydrogen, halo, hydroxy, nitro, cyano, C₁₋₄ alkyl, C₁₋₄ alkoxy, CF₃ or OCF₃.

7. A compound as claimed in claim 1 wherein R⁶ is C₁₋₄ alkyl and wherein the S(O)₂R⁶ group of formula (I) is para disposed to the remainder of the structure of formula (I).

8. A process for the preparation of a compound as claimed in claim 1, the process comprising:
a) reductive amination of a compound of formula (II) or (IIa): 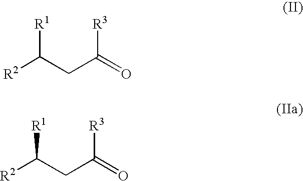
with a compound of formula (III): 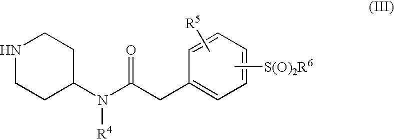
in the presence of NaBH(OAc)₃ (wherein Ac is C(O)CH₃) and acetic acid, in a suitable solvent at room temperature; or,
b) coupling a compound of formula (IV) or (IVa): 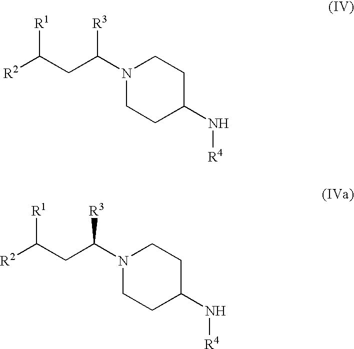
with a compound of formula (V): 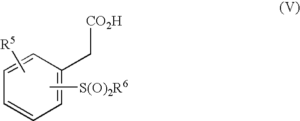
in the presence of a suitable coupling agent, in the presence of a suitable base, in a suitable solvent and at room temperature.

9. A pharmaceutical composition which comprises a compound as claimed in claim 1, or a pharmaceutically acceptable salt thereof or solvate thereof, and a pharmaceutically acceptable adjuvant, diluent or carrier.

10-11. (Cancelled)

12. A method of treating a CCR5 mediated disease state comprising administering to a patient in need of such treatment an effective amount of a compound as claimed in claim 1.

13. The method of claim 8 wherein the temperature during step b) is from 10° C. to 30° C.
